(19) United States
(12) Patent Application Publication
(10) Pub. No.: US 2016/0146803 A1
(43) Pub. Date: May 26, 2016

(54) HETEROGENOUS ASSAY

(71) Applicant: CARCLO TECHNICAL PLASTICS LIMITED, Yorkshire (GB)

(72) Inventors: Gerald John Allen, Caythorpe (GB); Carolyn Jennifer Ruddell, Wirral (GB); Patrick Ward, Croydon (GB); Philip Roberston, Weybridge (GB)

(21) Appl. No.: 14/763,084

(22) PCT Filed: Jan. 27, 2014
(86) PCT No.: PCT/GB2014/050198

§ 371 Date: Jul. 23, 2015

(30) Foreign Application Priority Data
Jan. 25, 2013 (GB) 1301333.9
Aug. 6, 2013 (GB) 1314057.9

(51) Int. Cl. G01N 33/543; B01L 3/00

(52) U.S. Cl. CPC G01N 33/54386; B01L 3/561; B01L 2300/0838; B01L 2300/0636; B01L 2200/0621

(57) ABSTRACT

The invention relates to a sample testing device for conducting a heterogenous assay, for example an ELISA, in a capillary lumen, using one way flow of sample and wash buffer to move the reaction through the binding, separation and signal measurement steps, thus minimising external intervention. The capillary passage is configured to allow time within different zones for reaction, capture, separation of bound and free fractions, and signal measurement. A combined capture-signal read zone is provided to maximise the capture of signal linked binding member, and signal measurement within the capture zone.

The present invention relates to a sample testing device for conducting a heterogenous assay, for example an ELISA assay, in a capillary lumen. The present invention also provides a method of conducting a heterogenous assay, for example an ELISA assay, in a capillary lumen of a sample testing device. Also provided is a combined capture and signal-measurement zone for use in combination with a sample testing device.

### BACKGROUND

The Point-of-Care (PoC) sector encompasses all assays performed in a non-laboratory setting, including satellite laboratories in hospitals, A&E departments, ambulances, doctor's surgeries and homes. PoC assays are becoming increasingly important for in vitro diagnostics (IVD) because of the advantages they offer, particularly with regard to the time from patient sampling to result. By obtaining early results, clinical decisions can be made more rapidly, and suitable treatment can be initiated earlier or therapy adjusted. This result in overall cost savings by releasing patients sooner, avoiding inappropriate therapy, and improving patient outcomes.

Currently, the immunoassay PoC sector is dominated by tests utilising membrane-based lateral flow technology (LFT), as exemplified by the widely-known pregnancy test. With these tests, all of the reagents necessary for performing the test are positioned along a bibulous strip. Patient sample (e.g. urine) is added to one end of the membrane and flows along the strip by capillary action, reconstituting reagents as it passes and reacting with them. The label is usually a chromophoric particle (e.g. gold sol, coloured latex). In the presence of analyte, the signal reagent becomes bound to an immobilised antibody capture zone. Although these tests meet some of the requirements for PoC tests (e.g. low cost, can be performed by non-skilled personnel, are self-contained, etc) they are primarily qualitative (yes/no) tests. However, relatively few medical conditions can be diagnosed or monitored by a qualitative assay. The majority require a quantitative estimation of the level of a biomarker specific for the disease, or detection of an increase/decrease in the level of analyte.

Although attempts have been made to quantify lateral flow technology assays using readers to measure the immobilised signal (usually reflectometers), the drawbacks of the technology frequently result in poor precision and reduced sensitivity. The main problems arise from the use of bibulous membranes as the capillary matrix as they have inherently variable fluid flow and it is difficult to accurately control the fluid. Fluid control is a pre-requisite for precise, controlled assays.

Sensitivity in immunoassays is in part dependant on signal intensity. The higher the intensity of the signal, the greater the assay sensitivity. A variety of labels have been employed in known assays, including radionuclides, and fluorophores. However, these typically require the use of sophisticated instrumentation for their measurement.

An alternative approach has been to use an amplification system to generate a signal that can be measured using relatively simple detection systems. Enzyme-linked immunosorbent assays (ELISA) are analytical tools for determining the presence, absence, or amount of analyte in a sample. There are several formats of ELISA but all are based on the same underlying principle, namely that one component of the reaction is labelled (i.e. coupled to) with an enzyme which can act upon a substrate to generate a coloured signal which is related to analyte concentration. As measurement of colour only requires a relatively simple instrument, the cost and complexity are reduced yet assay sensitivity is maintained by virtue of the signal amplification. The 2-site assay format (or sandwich ELISA) is based upon using a first binding partner immobilised on a solid phase to capture analyte from a sample, and using a second binding partner with enzyme attached thereto, to bind to the captured analyte. The enzyme causes a colour change upon reaction with its substrate, which is added in a final step of the assay, such that the intensity of colour produced is directly proportional to analyte concentration. A competition assay format typically employs an immobilised binding reagent in conjunction with an enzyme-labelled analyte-analogue which competes with analyte for binding sites on the immobilised binding reagent. When substrate is added, the colour generated by enzyme action upon substrate is inversely proportional to the analyte concentration. Other formats include the 1-site immunometric assay, specific antibody tests using immobilised analyte analogue, and antibody class capture assays (ACCA).

ELISA's have become a widely adopted in IVD, facilitating quantitative assays with high sensitivity and specificity. However, these assays require a complex protocol with multiple reagent additions and separations (wash steps) for the various stages of the assay. Accurate volume additions and precise timing of steps is essential if accurate and reproducible results are to be obtained. This either requires skilled operators and laboratory equipment, or expensive fully-automated assay systems. Because of this, they have not been widely adopted for the Point-of-Care (PoC) segment of the IVD market, where the requirement is for simple protocols which can be performed by unskilled staff with no equipment and which are fool-proof.

Disposable devices have been disclosed that include some features of an integrated system, but none include all the features necessary for performing a quantitative fully-integrated device for performing immunoassays.

U.S. Pat. No. 5,837,546 (Allen et al, Metrika) describes a fully-integrated system based on lateral flow technology with an in-built reflectometer and data reduction capability. The system uses chromogenic particles as signal, and has no capability for performing assays based on signal amplification (e.g. enzyme labels used in conjunction with a substrate). The read-out is an LCD screen, so the output is only transiently readable whilst the battery has capacity to power the device.

Because of the drawbacks with current systems, heterogenous assays are still primarily performed in centralised laboratories. There exists therefore a requirement for a low-cost, self-contained system which can deliver quantitative results with minimal operator intervention or skill.

The present invention aims to overcome or ameliorate problems associates with the prior art.

### BRIEF SUMMARY OF THE INVENTION

In a first aspect of the invention, there is provided a sample testing device for performing a heterogeneous assay, wherein the device comprises:

(i) a capillary passage having a lumen;
(ii) a combined capture and signal measurement zone fluidly connected to the capillary passage; and
(iii) an optical pathway across the combined capture and signal measurement zone; wherein the combined capture and signal measurement zone includes a plurality of elongate fins projecting substantially perpendicularly from a base, where each elongate fin has a length that is substantially parallel to the base, the elongate fins being arranged so that:
the lengths of the plurality of elongate fins are substantially parallel to one another;
the plurality of elongate fins are aligned along a line that is substantially perpendicular to the lengths of the fins; and
the lengths of the plurality of elongate fins are substantially perpendicular to said optical pathway;
said plurality of elongate fins permitting optical transmission therethrough along said optical pathway and defining a plurality of fluidic channels therebetween along the base for receiving fluid from said capillary pathway.

The sample testing device may comprise a capillary passage having a lumen, and serving to fluidly connect in series:

(i) a fluid application region at an upstream end of the capillary passage;
(ii) a reagent zone;
(iii) a combined capture and signal measurement zone, wherein the combined capture and signal measurement zone comprises means for directing an optical pathway across the combined capture and signal measurement zone; and wherein the combined capture and signal measurement zone includes a plurality of elongate fins projecting substantially perpendicularly from a base, where each elongate fin has a length that is substantially parallel to the base, the elongate fins being arranged so that:
the lengths of the plurality of elongate fins are substantially parallel to one another;
the plurality of elongate fins are aligned along a line that is substantially perpendicular to the lengths of the fins; and
the lengths of the plurality of elongate fins are substantially perpendicular to said optical pathway;
said plurality of elongate fins permitting optical transmission therethrough along said optical pathway and defining a plurality of fluidic channels therebetween along the base for receiving fluid from said capillary pathway; and
   (iii) an outlet and/or fluid sump.

The capillary passage may be designed for one way flow of sample, from the fluid application region toward the outlet and/or fluid sump. By provision of reagent in the reagent zone, the device is suitable for conducting a heterogeneous assay without the need for external steps, for example addition of reagent. The capillary passage is designed to allow for sufficient time for each stage of a heterogeneous assay to take place during flow from the fluid application region toward the outlet and/or fluid sump. Thus, the length of capillary passage which fluidly connects the reagent zone and capture zone (referred to as a reaction zone) is determined by the time required for reaction between sample and reagents. Knowing the time required, a skilled person can calculate the necessary minimal dimensions of the capillary passage of the reaction zone.

Similarly, the length of capillary passage which fluidly connects the capture zone and the outlet and/or fluid sump at a downstream end of the capillary passage may be referred to as a wash zone. The dimensions of the capillary passage defining the wash zone determines, at least in part, the amount of washing e.g. the volume of wash buffer, and/or the time allocated for washing. Thus, by knowing the amount of time or the volume required for washing a skilled person can calculate the necessary dimensions of the capillary passage of the wash zone.

The capillary passage may comprise a widened portion for housing the combined capture and signal measurement zone, to aid flow along the capillary passage and through the combined capture and signal measurement zone. The capillary passage may widen immediately upstream and/or immediately downstream of a capture and/or signal measurement zone in the capillary passage, such that the sides of the capillary passage align with the sides of a capture and/or signal measurement zone. Thus, in combination the widened portions and combined capture and signal measurement zone form a widened portion with elongate sides, with the capture and signal measurement zone extending across the portion, perpendicular to the elongate sides. The widened portion may be an oval, trapezoidal or diamond shaped portion. The widened portion allows for a larger optical window.

All or part of a widened portion may comprise microstructures (for example, micropillars), to aid flow of liquid across the combined capture and signal measurement zone. Preferably, microstructures are provided immediately upstream and/or downstream of a capture zone, or combined capture and signal measurement zone. In an embodiment, the micropillars are elongated in cross section. In an embodiment, the micropillars project from the base and are elongated, where one dimension of each micropillar exceeds a perpendicular dimension of the micropillar in the cross section that is parallel to the plane of base. Preferably, the longer direction of each micropillar is orientated substantially parallel to the intended direction of flow of liquid across the combined capture and signal measurement zone.

The capillary passage may be arranged relative to the plurality of elongate fins to permit sequential flow through the plurality of fluidic channels. In an embodiment, the capillary passage fluidly connects adjacent individual fluidic channels so that said sequential flow occurs through individual ones of the plurality of fluidic channels. This is in contrast to the embodiment where a widened portion of a capillary passage is provided as described above, where the formation of the capillary passage allows for simultaneous flow through each of the fluidic channels. In this embodiment for sequential flow, the capillary passage includes a series of looped portions that direct fluid travelling along the capillary passage sequentially through adjacent fluidic channels defined by the fins. Looped portions may extend alternately upstream and downstream. The looped portions of the capillary passage may form a single fluidic pathway, which provides a fluid path between adjacent fluidic channels. Downstream, the capillary pathway provides a fluid path away from the signal measurement zone.

One or more of the fins may be formed as an insert for integration with the device, or they may be formed integrally with one or more other components of the sample testing device. In such an embodiment, without the fins present, the device comprises an open space (or cavity) between the reagent zone and wash zone. Thus, it may include a series of disjointed looped portions, which together with one or more inserted fins forms a capillary passage, for example of serpentine configuration.

Where the capillary passage provides for sequential flow through the fluidic channels it provides a longer path length for the fluid and so increases contact time with the fins, and may improve washing by minimising so-called “dead-spaces”, where adequate mixing and reaction does not occur

A combined capture and signal measurement zone may comprise means to capture bound fraction of signal linked binding member. A capture zone may comprise a member of a binding pair, for example applied to a surface thereof. The captured (“bound”) fraction of signal linked binding member is directly or indirectly proportional to the amount of analyte in the sample. The member of a binding pair may be an analyte-specific receptor, such as an antibody or antigen.

Alternatively, a binding member may be linked to the surface of the capture zone, for example by use of a biotin-labelled binding member and streptavidin or avidin immobilised on the surface of the capture zone.

The device may comprise a second capture zone, for example for retaining or capturing the “free” fraction of signal linked binding member (i.e. that fraction which was not captured in the first capture zone). Measurement of the “free” fraction in a second capture zone may be useful in the measurement of the amount of analyte.

The assay is preferably an ELISA assay. In such an embodiment, the signal is an enzyme.

The sample testing device may comprise means for metering a volume of sample. Thus, a sample testing device of the present invention may comprise a first inlet at an upstream end of the capillary passage, and which is fluidly connected to the fluid application region. A second inlet is provided, to enable the application of a buffer or other non-sample fluid to the capillary passage, after the sample.

The device may comprise a second capture zone for example for control or correction of results, (for example, for capture of a “free” fraction (the signal linked binding member which is not captured in the first capture zone).

The sample testing device may comprise flow control means, preferably in the form of outlet sealing means. Flow control means may be optionally provided on a control element.

The sample testing device may comprise fluid dispensing means.

The sample testing device may comprise signal processing means.

The sample testing device may comprise a display.

In a second aspect of the invention, there is provided a method of performing a heterogeneous assay in a capillary lumen of a sample testing device, for detection of analyte in a sample, wherein the method comprises the steps of:

(a) providing a sample testing device comprising:
(I) a capillary passage having a lumen, and serving to fluidly connect, in series:
i. a fluid application region at an upstream end of the capillary passage;
ii. a reagent zone comprising a signal-linked binding member;
iii. a capture zone comprising means to capture the signal linked binding member (a “bound” fraction);
   (b) adding sample to the fluid application region and causing it to flow downstream by capillary action through the reagent zone, thus creating a mixture of sample and reagent including signal linked binding member;
   (c) adding a wash buffer and causing it to flow downstream in the capillary passage following the sample, such that any sample or reagent which is not retained by the capture zone (the “free fraction”) passes downstream through the capture zone;
   (d) detecting any signal of the captured signal linked binding member in the capture zone as a measure of the amount of analyte present in the sample.

Preferably, the capture zone is also a signal measurement zone, for example a combined capture and signal measurement zone, for example as described herein.

The method of the invention has the advantage that all steps of a heterogeneous assay are performed within a single capillary passage of a device, during one way flow from one end of the capillary to the other. Thus, external operator steps are minimised.

Preferably, the method comprises providing a device of the first aspect. As discussed above, such a device may be configured such that dimensions of the capillary passage in the reaction and wash zones allow sufficient time for reaction and/or separation to take place.

The device may comprise a second capture zone, which may be used for assay control purposes, or for correction or normalisation of results to compensate for variation in ambient temperature, reagent degradation on storage or shipping, etc. Thus, the method may comprise the step of capturing the “free” fraction (the signal linked binding member which is not captured in the first capture zone). The method may comprise the step of measuring the amount of signal linked binding member in the second capture zone. The method may comprise the step of measuring the total amount of signal bound to both capture zones and calculating the percentage of the total signal captured by the first or second or both capture zones.

The method of the invention may include any heterogeneous assay, including measurement of direct signal (e.g. where signal is not amplified such as coloured particles or fluorescence based assays) and generated signal, e.g. where signal is developed and/or amplified, for example by a catalyst or enzyme.

The assay is preferably an ELISA assay. In such an embodiment, the signal is an enzyme. The method may comprise the step of providing to the capture zone a substrate for the enzyme. The substrate may be provided to the capture zone prior to detection of the signal; and more preferably, with or subsequent to the wash buffer.

Where the signal is an enzyme or catalyst, the reaction predominantly takes place in the capture zone, where signal linked binding member is retained.

The signal may be an enzyme. In an embodiment, the enzyme substrate may be provided in the wash buffer or as a separate substrate solution. The enzyme may cause a change in the substrate, which is detected in the capture zone. For example, the change may be a change in colour of the substrate, which may be detected by any suitable method, for example light absorption. Alternatively, the enzyme or catalyst may react with the substrate to generate a fluorescent compound, which may be detected by any suitable means. In an embodiment, excitation light may be directed through the capture zone, and the fluorescence detected.

The method may comprise providing a sample testing device comprising a combined capture and signal measurement zone. In an embodiment, the combined capture and signal measurement zone may comprise means for directing an optical pathway across the combined capture and signal measurement zone. In an embodiment, the combined capture and signal measurement zone includes a plurality of elongate fins projecting substantially perpendicular from a base, where each elongate fin has a length that is substantially parallel to the base, the fins being arranged so that:
the lengths of the plurality of elongate fins are substantially parallel to one another;
the plurality of elongate fins are aligned along a line that is substantially perpendicular to the lengths of the fins; and
the lengths of the plurality of elongate fins are substantially perpendicular to said optical pathway;
said plurality of elongate fins permitting optical transmission therethrough along said optical pathway and defining a plurality of fluidic channels therebetween along the base for receiving a fluid from said capillary pathway.

In a third aspect of the invention, the present invention provides a kit comprising
i) a sample testing device comprising a capillary passage having a lumen;
ii) a combined capture and signal measurement zone including a plurality of elongate fins projecting substantially perpendicular from a base, where each elongate fin has a length that is substantially parallel to the base, the fins being arranged so that:
   the lengths of the plurality of elongate fins are substantially parallel to one another;
   the plurality of elongate fins are aligned along a line that is substantially perpendicular to the lengths of the fins; and
   the lengths of the plurality of the elongate fins are substantially perpendicular to said optical pathway;
   said plurality of elongate fins permitting optical transmission therethrough along said optical pathway defining a plurality of fluidic channels therebetween along the base for receiving fluid from said capillary pathway.

The sample testing device and combined capture and signal measurement zone may be provided as separate components in a kit, for assembly by a user.

The capillary passage may comprise a widened portion into which combined capture and signal measurement zone is inserted. Alternatively, the capillary passage does not form a continuous fluid path and instead includes a series of disjointed looped portions. When the combined capture and signal measurement zone is inserted, the looped portions of the capillary passage and the fluidic channels between adjacent fins together form a single fluidic channel, for example of serpentine configuration. The embodiments described in relation to the first aspect, apply also to this aspect.

Thus, a capillary passage of a sample testing device of a kit may be disjointed, comprising two or more separate portions which upon insertion of the combined capture and signal measurement zone, form a single fluidic channel.

A kit may alternatively comprise a sample testing device according to the first aspect of the invention, instructions for use and a control sample.

A kit may additionally comprise, materials and apparatus mentioned herein such as buffers, fluid filled capsules, detectable particles, application means (for example pipettes), instructions, charts, desiccants, control samples, dyes, batteries, signal processing means and/or display means.

In a fourth aspect, there is provided a combined capture and signal measurement zone, wherein the combined capture and signal measurement zone comprises means for directing an optical pathway across the combined capture and signal measurement zone; and wherein the combined capture and signal measurement zone includes a plurality of elongate fins projecting substantially perpendicular from a base, where each elongate fin has a length that is substantially parallel to the base, the fins being arranged so that:
the lengths of the plurality of elongate fins are substantially parallel to one another;
the plurality of elongate fins are aligned along a line that is substantially perpendicular to the lengths of the fins; and
the lengths of the plurality of elongate fins are substantially perpendicular to said optical pathway;
said plurality of elongate fins permitting optical transmission therethrough along said optical pathway and defining a plurality of fluidic channels therebetween along the base for receiving fluid from said capillary passage.

### DESCRIPTION OF THE DRAWINGS

Embodiments of the invention are further described hereinafter with reference to the accompanying drawings, in which:

FIG. 1 is a diagrammatic representation of a typical standard ELISA assay procedure, where numbers 1-11 in the schematic represent the following steps. 1. Preparation of reagents and samples, 2. Addition of samples and calibrators to microtitre plate, 3. Incubation at room temperature for 1 hour (to allow binding of analyte to plate via capture antibody), 4. Washing of microtitre plate to remove unbound sample components (repeat 3 times), 5. Addition of HRP-labelled signal antibody to plate, 6. Incubation at room temperature for 30 mins (to allow binding of signal antibody to analyte), 7. Washing of microtitre plate to remove unbound signal antibody (repeat 3 times), 8. Addition of TMB chromogenic substrate to plate, 9. Incubation at room temperature (in darkness) to allow signal to develop, 10. Addition of stop solution to halt reaction and convert chromogen from blue to yellow colour, 11. Quantitation of signals using a spectrophotometer at 450 nm;

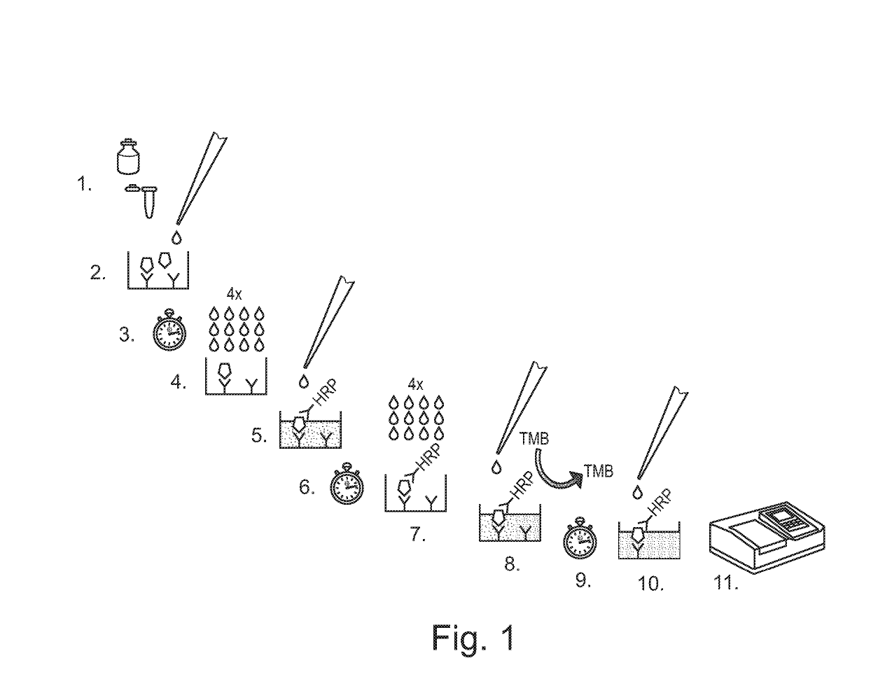

FIG. 2 is a diagrammatic representation of a capillary based heterogenous assay;

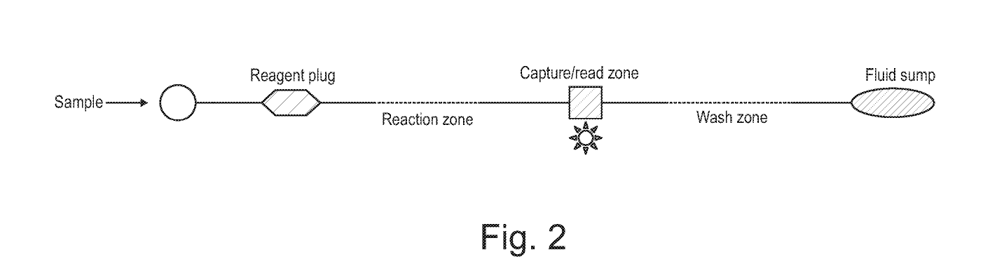

FIG. 3 shows a cross section through a sampling testing device having a finned section across the light path, and micropillars either side thereof;

FIG. 4; shows an embodiment of a combined capture and signal measurement zone;

FIG. 5 shows a plan view of the underside of a sample testing device, showing a capillary passage and side passage for sample metering;

FIG. 6 shows a perspective view of a device of the invention with a control element;

FIG. 7 shows fluidic control aspects of a device of the invention from above;

FIG. 8 shows a perspective view of a device of the invention with fluid dispensing means;

FIG. 9 shows assembly of a control element;

FIG. 10 is a detail of a combined capture and signal measurement zone;

FIG. 11 shows transmittance spectra of TMB and enzyme over time;

FIG. 12 shows absorbance of TMB and enzyme reaction over time at 3 wavelengths;

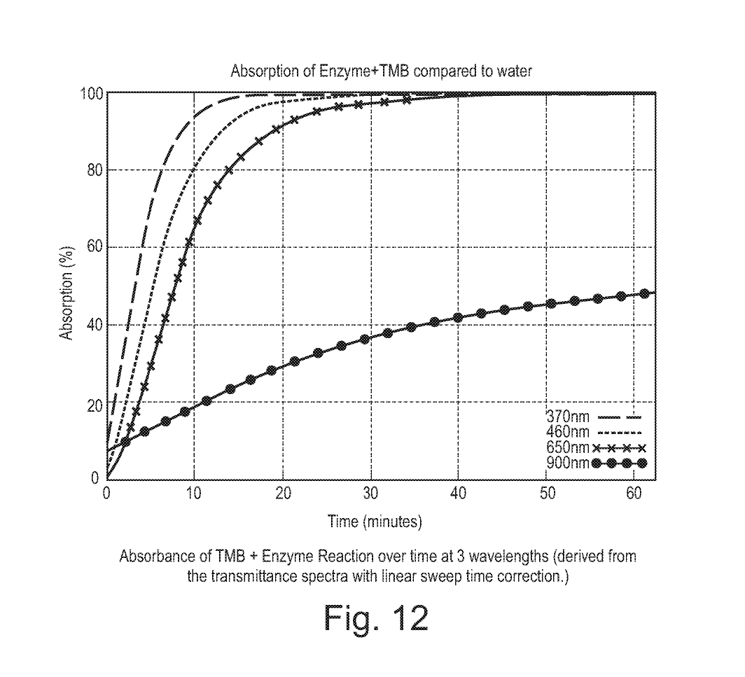

FIG. 13 shows reflection of TMB and enzyme reaction over time;

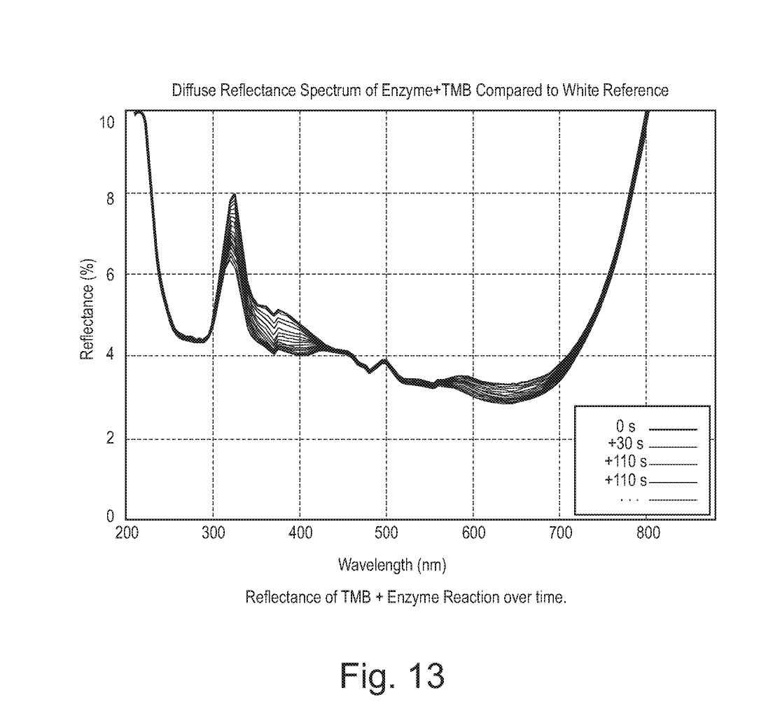

FIG. 14 shows the signal obtained at 370 nm using a spectrophotometer over 30 minutes development time;

FIG. 15 shows the results of a simultaneous fluid phase immune reaction, measured at 370 nm over 30 minutes development time;

FIG. 16 shows a typical Optical Transmission Curves for 2 wavelengths;

FIG. 17 shows a part of a device of the invention, where a fluid sump adjoins and overlies a fluid outlet; FIG. 17B shows the fluid sump with the absorbent pad;

FIG. 18 shows a perspective view of an embodiment of a capillary pathway device in accordance with an aspect of the present invention;

FIG. 19A shows a detailed view of the combined capture and signal measurement zone of the device of FIG. 18; and

FIG. 19B shows the detailed view of FIG. 19A with the finned insert of the combined capture and signal measurement zone removed.

FIG. 20 shows a dose-response relationship between pi-GST concentration and assay signal (rate of generation of blue colour at 632 nm) (Example 6).

FIG. 21 shows the underside of a device with consecutive fluid inlets and a spiral fluid sump.

FIG. 22 shows a device with a serpentine capture/signal measurement zone.

### DETAILED DESCRIPTION OF THE INVENTION

The present invention has the advantage that it provides a sample testing device and method for performing a heterogeneous assay in a capillary passage having a lumen, using one way flow of sample and wash buffer to move the reaction through the necessary binding, separation and signal measurement steps, thus minimising external intervention. The ability to perform a heterogeneous assay in a capillary passage lumen is enabled by the provision of a sample testing device comprising a capillary passage whose dimensions are configured to allow sufficient time within different zones for reaction, capture, separation of bound and free fractions, and signal measurement. Preferably, the passage comprises a combined capture-signal read zone which is designed to maximise the capture of signal linked binding member, whilst allowing separation of unbound signal linked binding member, and enabling signal measurement within the capture zone.

The device of the present invention enables a heterogeneous assay to be conducted in a point of care environment, by unskilled persons. It may either have the advantage of giving a permanent or semi-permanent readout. The invention is particularly suited to performance of an ELISA assay, but can equally be applied to a variety of other heterogeneous assays.

A combined capture and signal measurement zone of the present invention has the advantage that it addresses the problem of different competing requirements. Specifically, for efficient and rapid capture the requirement is for as large a surface area as possible, with a maximal surface:volume ratio. For rapid and efficient washing, a smooth surface with minimal “dead zones” is required. To maximise sensitivity of measurement, the signal is preferably concentrated in minimal volume. A combined capture and signal measurement zone of the present invention offers a design which is able to satisfy these conflicting requirements of the different assay activities within a single zone.

The prior art has made attempts to resolve this issue, but the majority (as exemplified by Allen/Metrika, supra) use a porous strip, with a capture zone through which the fluid flows for washing and where the signal accumulates. However, these systems are not ideally suited for enzyme-linked signal systems (where signal needs to accumulate in a constrained, defined volume) and require a reflectance measurement to be made. Such measurements in a porous strip are less accurate and reproducible as they can be influenced by variations in the underlying substrate (variations in reflectivity, uneven surface can scatter light, etc) and the reflectivity can be adversely affected by variable drying of the substrate (e.g. nitrocellulose is white when dry, translucent when wet; see U.S. Pat. No. 4,025,310, International Diagnostic Technologies). Other systems (e.g. Biosite Triage, Response Biomedical RAMP) similarly use reflectance measurements but are based on the use of a separate chip and reader.

The present invention is particularly suited for use in assaying a sample liquid for a particular component. Whilst it may be suited to biological and non-biological applications, it is particularly suited to the former. Thus, the present invention is preferably for use in assaying a biological sample for a particular component, for example an analyte, using a heterogeneous assay, for example an ELISA assay. The assay may be quantitative or qualitative, preferably quantitative. The present invention may be suitable for use with any liquid or fluid sample. Preferred samples for assay using the present invention are blood (whole blood or serum/plasma) and urine. Herein, the terms liquid and fluid may be used interchangeably.

The invention finds particular application in sample testing devices having one or more capillary passages for testing for the presence of a component of interest in a liquid sample, e.g. blood or serum/plasma or other body fluid, as is well known in the art, e.g. diagnostic assays.

The sample testing device may comprise a moulded plastics component, e.g. in the form of a generally planar element having grooves in one surface thereof to define a capillary passage having a lumen, when sealed by a cover member. Capillary passages having a lumen formed in other ways are also included.

The present invention is typically applicable to a sample testing device in which fluid flow is passive, i.e. it is not reliant upon an external propulsive force.

A heterogeneous assay is defined as an assay that incorporates a signal system and where a bound and unbound fractions of signal-linked binding member are separated prior to measurement of a signal. A heterogeneous assay may be an ELISA assay, for example a competition or sandwich ELISA assay.

### Capillary Passages

The sample testing device comprises a capillary passage having a lumen. A capillary passage is a tube, which comprises a lumen. A capillary passage of the sample testing device may fluidly connect, in series, zones or stations for performing one or more steps of an assay. A capillary may be formed as a groove, moulded in a planar thermoplastic chip, sealed by a foil or sheet to form the lumen. Any suitable thermoplastic may be used including, but not limited to, polystyrene, polycarbonate, ABS, etc. Preferably, polycarbonate is used. Any suitable foil or sheet can be used to complete the capillary. Preferably a thin foil of polycarbonate is used. The foil or sheet can be sealed to the chip by any means, including adhesives, ultrasonic welding, laser welding, etc. The use of laser welding is preferred as it gives a controllable seal and avoids the use of adhesives which may interfere with the reagents and/or flow characteristics of the device. Other methods of forming the capillary passage are included within the scope of the invention and are known to persons skilled in the art.

If a hydrophobic material is used, such as polycarbonate, it may be desirable to treat the surface to ensure uniform and consistent flow characteristics. Any suitable treatment can be employed, such as plasma treatment, corona discharge, surfactants and the like. Surfactants are preferred, for example Tween-20. Alternatively, components may be incorporated into the formulation of the material before molding to reduce hydrophobicity.

A capillary passage may have any suitable geometry, typically dictated by the type. It may be linear. All or part of a passage may be straight, curved, serpentine, spiraled, U-shaped, etc. A capillary passage comprising a serpentine configuration through all or part of a capture and/or signal measurement zone is preferred. A capillary passage having a fluid sump in the form of a spiraled capillary passage may be preferred.

The cross-sectional configuration of a capillary lumen may be selected from a range of possible forms, e.g. triangular, trapezoidal, square, rectangular, circular, oval, U-shaped, etc. Most preferred is a V-section as this is suitable for economic and consistent manufacture, and such a shape has been found to promote effective mixing of sample and reagent and to exert a strong capillary “pull”. By careful selection of materials, capillary shape, surface treatment, seal and sealing means it is possible to produce a capillary which facilitates even and consistent fluid flow, with good reproducibility between devices, without the requirement for any additional or external sources of fluid propulsion.

A capillary passage may have any suitable dimensions. A capillary passage referred to herein is microfluidic. Typical dimensions of a capillary passage for use in the invention is a lumen depth of 0.1 mm to 1 mm, more preferably 0.2 mm-0.7 mm. The width of a lumen may be of similar dimensions to the depth. Where the lumen is V-shaped, for example, the profile may be that of an equilateral triangle, each side having a length of between 0.1 and 1 mm, more preferably between 0.2 and 0.7 mm.

The dimensions of each zone of a capillary passage will dictate the volume of reagent or buffer required; the dimensions and shape will dictate the reaction time for that zone (e.g. curves slow flow). Dimensions may be readily calculated by a person skilled in the art, based upon knowledge of the reaction time required.

Each capillary passage may consist of one or more capillary segments, joined to form a pathway from a fluid application region to an outlet. Segments of capillary passage may be interposed with a section selected from a capture zone, a signal measurement zone, a combined capture and signal measurement zone, a reagent zone, a reaction zone, a wash zone, a fluid application region, and an outlet and/or fluid sump. Any of these sections may have a shape and configuration different to the capillary segment to which it is adjoined.

In the present invention, a device may include more than one (i.e. two, three, four, five or more) capillary passages, preferably one or more being as described herein.

Where more than one capillary passage is provided in a device, the geometry and dimensions of each may be independently selected, and two or more may be the same or different. Two or more capillary passages may be connected to a common fluid application region or outlet/sump.

Preferably, each capillary passage is fluidly connected to a first inlet, for introduction of sample to the capillary passage, and an outlet and/or sump.

In an embodiment, a capillary passage of the invention may fluidly connect, in series, a reagent zone, a reaction zone, a combined capture and signal measurement zone, a wash zone and a fluid sump. Preferably, the capillary passage is fluidly connected to a fluid application region at an upstream end. Preferably, the capillary passage comprises an inlet for sample, upstream of the reagent zone, and an outlet at, or downstream of, the fluid sump.

Thus, in combination the widened portions and combined capture and signal measurement zone form a widened portion with elongate sides, with the capture and signal measurement zone extending across the portion, perpendicular to the elongate sides. The widened portion may be an oval, trapezoidal or diamond shaped portion. The widened portion allows for a larger optical window.

A capillary passage may comprise parts or sections which are not in the form of a capillary passage, or may be interrupted by such sections. For example, a capillary passage may widen immediately upstream and/or downstream of a capture and/or signal measurement zone, such that the sides of the capillary passage align with the sides of the capture and/or signal measurement zone to smooth flow between these sections. This may be the case where a capture and/or signal measurement zone is not in the form of a capillary passage, but comprises a plurality of fluidic channels fed simultaneously by a capillary passage. Thus, an open mouth of a capillary passage immediately upstream and/or downstream of a capture and/or signal measurement zone may be widened or tapered, for example defining a triangular or semi-circular portion. The upstream and downstream widened portions may be the same shape or different, but preferably the capture and/or signal measurement zone and capillary passage immediately upstream and downstream is symmetrical about the optical pathway.

All or part of a widened portion may comprise microstructures (for example, micropillars), to aid flow of liquid, for example across a capture and/or signal measurement zone and minimise formation of bubbles. Preferably, microstructures are provided immediately upstream and/or downstream of a capture zone, or combined capture and signal measurement zone. Microstructures include for example micropillars, or roughened sections of capillary, bumps, lines, hatches, etc. Suitable structures for aiding capillary flow through a non-capillary section interrupting a capillary passage will be known to persons skilled in the art. Micropillars are preferred. In an embodiment, the micropillars are elongated in cross section. The micropillars may project from the base and are elongated, where one dimension of each micropillar exceeds a perpendicular dimension of the micropillar in the cross section that is parallel to the plane of base. Preferably, the longer direction of each micropillar is orientated substantially parallel to the intended direction of flow of liquid across the combined capture and signal measurement zone. The micropillars may be any suitable cross section, for example circular. Preferred micropillars have a height matching the depth of the capillary and a diameter of between 0.3 and 0.5 mm. Microstructures and micropillars are known in the art.

Widened or tapered portions may be provided in a capillary passage where appropriate, for example upstream and/or downstream of fluid application regions, sumps etc or any other non-capillary portion which interrupts the capillary passage. Microstructures as described herein may be provided in any one or more of these portions.

### Surface Treatment

A capillary passage of the device may be treated to improve flow of fluid therethrough, preferably by providing a surface coating on the internal surface of the passage. Any suitable method may be used, for example dip tweening or passing a treatment fluid through the passage followed by drying.

Thus, a capillary passage of the device may comprise a coating on the inner surface thereof, of a treatment fluid.

The coating may act by minimising any repulsion between the inner surface of a passage and sample or other fluid such as buffer, whilst preferably not actively binding or substantially reacting or binding therewith. The surface coating may increase the hydrophilicity of a passage, as compared to an untreated passage. The coating may, for example, act by forming a layer on the inner surface of the treated passage, polymerising with the surface of the treated passage, or soaking into the material of the treated passage. Preferably, it imparts hydrophilic properties.

A treatment fluid may be a liquid or a gas, but typically is a liquid. It may have suitable hydrophilic properties, e.g. a surfactant. Suitable materials are well known to those skilled in the art, and include for example bovine serum albumin, and polysorbates for example polyoxyethylene sorbitan materials known as Tween (Tween is a Trade Mark), e.g. Tween 20 (polyoxyethylene (20) sorbitan monolaurate), Tween 60 (polyoxyethylene (20) sorbitan monostearate), Tween 80 (polyoxyethylene (20) sorbitan monooleate). In an embodiment, a combination of BSA and tween is preferred. A treatment fluid may typically be used in the form of dilute aqueous solutions, e.g. 0.1 to 10%, typically. 1% by volume or less, typically in deionised water, although other solvents such as isopropanol (IPA) may alternatively be used.

Additionally or alternatively, a capillary passage or section thereof may be coated with, or may contain in a dissolvable form, a treatment to be imparted to the sample, such as anticoagulant or buffer. Preferably, a section of capillary upstream of the reagent zone is treated in this manner. Where a side passage is provided for reagent storage, a portion of capillary passage upstream of the intersection may be treated in this manner.

The thickness of the coating will depend upon the type of treatment fluid, the purpose of the coating, and the dimensions of the capillary passage. Where a layer of treatment fluid is left on the inner surface of the passage, it is preferably multi-molecular or mono-molecular layer. Preferably, substantially the entire inner surface (lumen) of the treated passage is coated with treatment fluid. Preferably, the lumen comprises an open-topped channel formed within a component, and the cover member thereof.

### Sample Well/Fluid Application Region

A fluid application region is an area designed to receive fluid, for example from a well, or directly from supply (e.g. a finger or pipette). An inlet may form part of an application region, or may be in fluid communication therewith, for example via a short passage. For example, an application region may be a widened section forming an entry to an inlet to which fluid or sample is applied, or may be part of a storage well. Thus, a fluid application region may form part of the sample testing device or may be separate thereto, for example as part of a control element which may be integrated with the sample testing device in an embodiment.

Herein, a fluid application region for receiving sample may be referred to as a sample application region. This may be fluidly connected to a first inlet, and/or a sample well. Any fluid application regions for receiving non-sample fluids may be referred to as fluid application regions. These may be each independently fluidly connected to second, third, fourth etc inlets.

Where two or more fluid application regions are provided, they may be provided in series, preferably at one end of the sample testing device.

A fluid application region may be an indented region, preferably conical-shaped, in a planar sample testing device. The indentation may penetrate the device, and fluidly connect to an inlet and/or capillary passage moulded into the underside of the device, for example as further described below. An inlet may be provided centrally to the application region, preferably centrally to an upstanding circular wall. A fluid application region may be any shape, but preferably is circular

A well may be provided, for holding sample or fluid, for application to a fluid application region. A separate well may be provided for each fluid which is to be provided in the assay, i.e. a sample well, a buffer well and/or a substrate well. Each well may be in fluid communication with a fluid application region, and therefore an inlet. A well may supply two or more capillary passages. A well may be any suitable shape and size, suitable for receiving and retaining liquid sample.

Each well may be independently formed within, or as part of, the sample testing device for example as a concave region leading to an inlet, or defined by a wall upstanding from the planar surface of the device, for example a collar. In these embodiments, the base of the well may comprise the fluid application region of the device. Alternatively, a well may be provided separately, i.e. it does not form an integral part of the device. Where provided separately it is preferably configured to fit with a fluid application region. All or part of the well may be provided as part of a control element as described herein. All or part of a well may consist of, or accommodate a capsule.

Where two or more wells are required, for example for supply of a sample to a first inlet and buffer and/or substrate to a second inlet, these may be independently provided either integral to the device or as a separable element, for example as described above. Thus, one or more wells may be provided as a separable element or control element and/or one or more wells may be provided as part of the sample element. In a preferred embodiment, at least a sample well and a fluid well are provided in a (one or more) separable element, preferably in a single separable element.

A well may be of any suitable size and shape. Preferably, a well is configured to aid drainage toward a fluid application region or inlet. For example, the base of a well may be funnel shaped, i.e. configured such that it slopes toward an inlet from all directions. This configuration aids drainage of sample or fluid into a capillary passage. Preferably a well comprises a suitable form of cap or cover, which is preferably removable, and may constitute one or more side walls of the well.

A cap of a well may comprise a liquid inlet for passage of liquid to the fluid application region, and thus the sample inlet.

A well may comprise features, for example microstructures for example micropillars, to aid liquid flow into a capillary passage. Suitable features will be known to a person skilled in the art.

### Sample Metering

The present invention may provide for sample metering of a sample. Thus, in an embodiment, sample metering means may be provided, which serve to provide a predetermined, measured volume of sample, or indeed other fluid, to a capillary passage for the assay. Any suitable sample metering means may be used, which may vary depending upon the form and purpose of the assay and device.

The device may comprise a side passage extending from a capillary passage part way along the length thereof and leading to a side passage outlet. The outlet of the side passage will be different to the outlet for the corresponding capillary passage.

Sample metering means in the form of a side passage with a side passage outlet may be used to provide a defined test volume of sample to the capillary passage. Preferably the intersection of the side passage with a capillary passage is downstream of a sample inlet, and any additional inlets, for example for buffer, substrate etc (referred to herein as second or third or further inlets).

When sample is provided to the fluid application region of the sample testing device, the capillary passage outlet is sealed, preferably by sealing means as described herein. The side passage outlet is not sealed. Sample may flow along the capillary passage by capillary action only as far as the intersection with the side passage, because the outlet of capillary passage is sealed. Sample is, however, able to flow into and along the side passage because the side passage outlet is not sealed. The capillary will fill until all sample has been drawn in. Any excess liquid above the test volume will begin to fill the side passage. Flow stops when all sample has been drawn in from the fluid application region into the capillary passage (the back pull in the capillary then equaling the forward pull). In this way, the capillary passage is filled with sample to a defined point (the intersection with the side passage). The volume of sample from the capillary passage inlet to the intersection with the side passage is referred to herein as a test volume. Any excess sample over the test volume is contained within the side passage. If the sample volume is too small, sample will not reach the side passage. Thus, it is preferred that sample in excess of the test volume is added to the device. Preferably, the test volume is a pre-determined volume, appropriate to the assay type. The conditions of sealing are then reversed, such that the capillary passage outlet is not sealed and the side passage outlet is sealed. The sample in the capillary passage is then free to flow further along the capillary passage, for example by capillary action. No further flow will take place along the side passage, including back-flow towards the capillary passage.

The mechanism has the advantage that the leading edge of the sample is not used as the test fluid, but is removed into a side passage as excess fluid. Thus the test volume of sample does not leave the capillary passage, and so can continue to flow along the capillary passage for the assay. No complex fluidics or additional sources of motive force are required other than capillary force. Further, the design is such that excess sample is contained safely within the device preventing any external contamination.

It may be advantageous to provide a second, in addition to a first inlet, and a capillary passage outlet; and a side passage extending from the capillary passage part way along the length thereof and leading to a side passage outlet.

Use of a second inlet, separate to the first inlet, is advantageous in those situations where a gross excess of sample is added to the device. In such situations, the side passage can become full while sample is still in the sample well. When e.g. wash buffer or substrate is introduced, sample can then enter then capillary leading to an excess of sample being introduced into the assay. The provision of a second inlet, downstream the first (sample) inlet neatly avoids this problem as e.g. wash buffer or substrate facilitates flow along the capillary of only the test volume and not excess sample. Further (third, fourth, fifth etc inlets) may be provided as appropriate. A second or further inlet is preferably provided in the same line of flow (i.e. connected in series) as the first inlet, upstream of an intersection of a capillary passage with a side passage.

A second, or further inlet is preferably located between the first inlet and the intersection with the side passage. The location of the second, third or further inlet determines the amount of sample test volume which is caused to move down the capillary passage by the application of fluid to a second inlet, as any sample between the first and second inlet will not form part of the test volume. Thus to maximise the test volume it is preferably located immediately downstream of the first inlet. Preferably, a second inlet is located within at least 15 mm, at least 7 mm or at least 5 mm of the first inlet. Preferably, a third inlet is within at least 15 mm, at least 7 mm or at least 5 mm of a second inlet, and so on.

Where two or more capillary passages are present, a second or further inlet can be provided separately for each capillary passage, downstream of a first inlet. Alternatively, it is envisaged that a common second, or further inlet may be shared between two or more passages, which may then be divided into separate passages. In such an embodiment, therefore, sample metering may take place in a shared portion of two or more passages. For any two or more capillary passages, it is preferred that a second, or further inlet is provided at a position such that the test volume drawn down the capillary passage in each capillary is the same. Thus, for example where the capillary passages have the same geometric dimensions in terms of width and height, the second, or further inlets will be provided at the same distance downstream from the first inlet, for each of said capillary passages. However, it is also envisaged that for any different two or more capillary passages in the same element, the test volume may be different, i.e. determined by a different positioning of the second inlet or junction with the side passage. Multiple similar capillary passages may be provided, e.g. for simultaneous testing of a single sample for multiple components of interest.

The size of the test volume depends on the cross-sectional area and length of the capillary passage between the most downstream fluid application inlet (typically the second, third or more inlet) and the side passage inlet. The size of the capillary passage between the second fluid application inlet and side passage inlet (the test volume) may be of any suitable size, depending upon the purpose of the assay. Preferred test volumes range from 1 to 200 l, more preferably between 1 and 150 l, more preferably between 1 and 50 l, more preferably between 1 and 20 l, more preferably between 1 and 10 l.

The side passage may also be a capillary passage, preferably a microfluidic passage. The side passage must be capable of capillary flow, but may adopt any configuration, not limited to that of a passage or tube. The size and shape of a side passage is typically dictated by the volume of sample it is required to accommodate. As the side passage is provided for storage of surplus sample, the same requirements of a test capillary passage, e.g. in terms of flow, reagent depositions, surface preparation, may not necessarily apply. The geometric and cross-sectional configurations of a side passage may be dictated by required volume to be held and the overall configuration of the device. The side passage may be wider or able to accommodate a larger volume than the test volume. For reasons including flow of sample, the side passage may be wider than the capillary passage. Preferably, the side passage has a volume of between 1 and 200 l.

Typical dimensions of a side passage for use in the invention is a depth of 0.1 mm to 1 mm, more preferably 0.2 mm-0.7 mm, most preferably approximately 0.5 mm. The width of a passage may be of similar dimensions to the depth. Typically, a side passage will have any length suitable depending upon the estimated sample size and the metering requirement, and also dictated by the shape and form of the device as a whole. Preferably, the side passage may have a length of between 20 and 100 mm, more preferably between 20 and 80 mm, more preferably approximately 60 mm.

A side passage may branch from a capillary passage in any direction, and may adopt any geometric configuration, for example it may be straight, curved, serpentine, U-shaped etc. It may extend parallel to a capillary passage to which it is fluidly connected, or perpendicular thereto. Preferably, a side passage is configured such that the side passage outlet is in close proximity to the capillary passage outlet, such that both may be operated by a single control element. The cross-sectional configuration may be any suitable configuration, for example trapezoidal, triangular, horizontal, square, rectangular, circular, over, or U-shaped etc.

Functionally, the configuration of a side passage must be such that it supports capillary flow, such that flow into the side passage can be remotely (i.e. without contacting the fluid) controlled by sealing or opening the side passage outlet.

A side passage may be treated to increase hydrophilicity, as described above in relation to the capillary passage.

### Inlets

An inlet is an entry hole. An inlet may be in fluid communication with a sample or fluid application region, preferably in direct fluid communication, so that fluid can enter a capillary passage. If in indirect communication, this is preferably via non-capillary passages or means. An inlet is positioned in a capillary passage at a suitable position from which fluid flow will start. Typically, this will be in close proximity to a well, or fluid flow control device which may be integrated with the device. Thus, an inlet may be downstream of a sample application region, but will be upstream of a reagent zone.

A device of the invention may comprise one or more (e.g. two, three, four or more) inlets, preferably each independently fluidly connected to a fluid application region. First, second, third or further inlets for sample or fluid application may be distinguished from other inlets of the device because they are each positioned to be in fluid communication with a fluid application region and where provided, a well which holds sample or other fluid.

A capillary passage may have one or more inlets and one or more outlets.

An inlet must be of a dimension which enables it to receive liquid. Preferably, for a sample testing device, an inlet will have an opening diameter in the region of 1 and 4 mm, preferably between 1 and 2 mm. For other applications, larger or smaller inlets are envisaged.

An inlet may have a raised skirt around the circumference, with the opening being central thereto.

Where two or more capillary passages are provided, a common first inlet may be provided, leading to or constituting the first inlets of two or more of the passages.

Herein the term “inlet” does not include openings sealed during manufacture.

A second, third or further (fourth, fifth, sixth etc) inlet may be provided in addition to a first inlet. Preferably, the inlets are all in the same line of flow (i.e. connected in series) as the first inlet.

A second, third or further inlet may each independently form part of a second, third or further fluid application region, which is in fluid communication with a well or other means for receiving and storing the fluid, for example a capsule. A second, third or further inlet may therefore be positioned and/or adapted for integration with a fluid flow control device comprising a well for storage and supply of fluid e.g. wash buffer. Preferably, a second, third or further inlet is supplied by its own well and fluid application region, which is separate from the fluid application region and/or well which supplies the first inlet.

In addition to a first and any second, third or further inlet of a capillary passage, a capillary passage may further comprise one or more additional inlets at one or more positions along the length of a capillary or side passage, for example for deposition of reagents in a passage or where branched (converging) channels or passages are provided. Typically, however, these additional inlets are sealed during manufacture and not operable or accessible by the user during performance of the test.

### Outlets

An outlet of a capillary passage or side passage is provided to enable flow through a passage, for example by capillary motive force, typically so that air can leave the passage.

An outlet may be provided at a distal end of a passage, although an outlet may be provided at one or more positions along the length of a capillary or any side passage. An outlet may not need to accommodate liquid flow therethrough. Preferably, it is able to accommodate air flow therethrough, sufficient to maintain flow of a fluid through the respective passage. An outlet may be of smaller dimensions than an inlet. An outlet may typically have an opening diameter of between 0.1 mm and 4 mm, more preferably between 0.3 and 2 mm. For other devices, larger or smaller outlets are possible. An outlet is typically only in fluid communication with a passage.

Outlets may have a raised skirt around the circumference, with the opening being central thereto.

Two or more outlets may be grouped together, for example so that they may be opened or closed by a single operation. Where a side passage is provided for sample metering, preferably the pair of outlets for the corresponding capillary passage and a side passage may be located within a close proximity so that they may be opened or closed by a single control element. Where two or more capillary passage are provided, each with a side passage, two or more side passage outlets may be grouped in close proximity, and two or more main capillary passage outlets may be grouped in close proximity, so that each group may be controllable by a single control element. Preferably, outlets or groups of outlets may be located in close proximity to a sample well or application region.

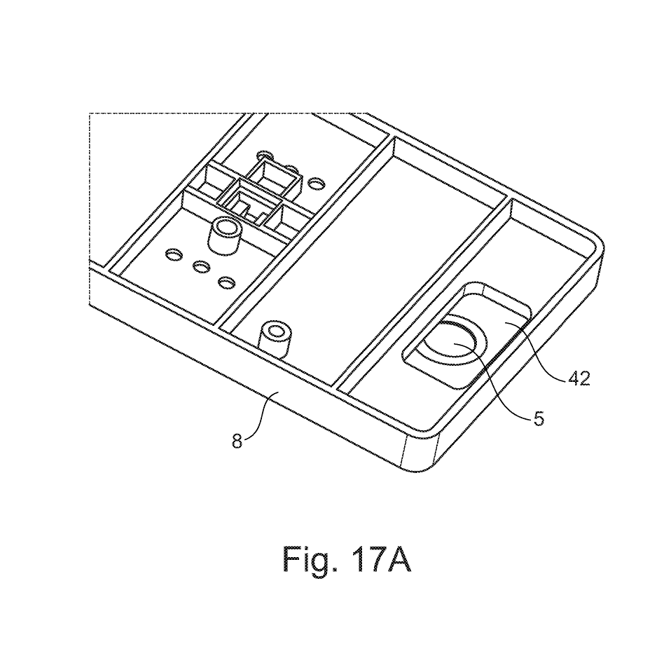

An outlet may adjoin and/or lie below a fluid sump, for example as shown in FIG. 17.

### Flow Control Means

It may be desirable to provide means to control flow in a capillary passage of a sample testing device of the invention. Flow control means may take any form, suitable to initiate, stop, resume or slow flow in a capillary passage. In an embodiment, the flow control means may be sealing means which open or close a capillary passage by acting as remote (off-line) valves, and so control passive flow of fluid through a passage of the device. Thus, sealing means may be releasably movable between a position in which the sealing means are positioned to seal an outlet and a position in which the outlet is not sealed, to stop or allow flow, respectively. By remote or off-line is meant that the valve (sealing means) is capable of controlling flow of a liquid sample (i.e. initiating, stopping, slowing, or resuming flow) without requiring contact between the sealing means and liquid sample. When a sample is provided via an inlet, sample will flow along the capillary passage only when the first sealing means is operated not to seal the outlet of the capillary passage. When the first sealing means is operated to seal the outlet, then fluid flow along the capillary passage is not possible. Thus operation of the sealing means can be used to control fluid flow in a capillary passage.

Sealing means may be provided externally to a passage, and therefore are capable of controlling flow of a liquid sample in the capillary passage without contact of the sealing means with the liquid sample. Thus, the sealing means are effectively off-line valves for control of sample flow, such that they are capable of controlling flow of a sample in a capillary passage without requiring contact between the sealing means and sample (i.e. they operate at a distance from the leading edge of the fluid).

Sealing means for use in the present invention must be sufficient to provide an air tight seal to a passage, when in a sealing relationship with an outlet. An air tight seal will substantially or completely stop fluid flow in the capillary passage to which the sealed outlet is related. Sealing means can be releasably operable.

In embodiments having two (or more) capillary passages, and/or one side passage, additional (second, third, fourth, fifth etc.) sealing means or components may be provided for releasably sealing a respective outlet of a second or further capillary passage, preferably conveniently located on a control element as discussed below. Thus, in a device comprising a second or further capillary passage, flow of sample in each passage is controlled by (preferably separate) first sealing means provided in respect of each passage.

Any sealing means may serve to seal one or more outlets. The outlets may be of capillary passages, side passages or a combination thereof. In an embodiment, a sealing means may operate to seal two or more capillary passage outlets, and a further sealing means may operate to seal two or more side passage outlets. Sealing means for a capillary passage outlet may be referred to as “first” sealing means and sealing means for a side passage outlet may be referred to as “second” sealing means.

In embodiments having two or more capillary passages, where one or more of said capillary passages having a side passage, one or more pairs of first and second sealing means may be provided. One or more pairs of sealing means may be constituted by a single sealing component. A sealing component may be provided on a control element. Such a component is moveable between a first position in which the first sealing means is positioned to seal the outlet of the capillary passage and the second sealing means is positioned not to seal the outlet of the side passage and a second position in which the first sealing means is positioned not to seal the outlet of a capillary passage and the second sealing means is positioned to seal the outlet of the side passage. In an embodiment, two or more first sealing means may be constituted by a single sealing component or provided on a control element. Two or more second sealing means may be constituted by a single sealing component or provided on a control element. A sealing component may be provided on a control element. Such a component or control element may be moveable between a first position in which the sealing means are positioned to not seal an outlet of a side passage and a second position in which the sealing means are positioned to seal an outlet of a side passage. In an embodiment, two or more first sealing means and two or more second sealing means, or two or more components may be provided on the same control element, which is moveable between a first position in which the first sealing means is positioned to seal the outlet of the first capillary passage and the second sealing means is positioned to not seal the outlet of the side passage; and a second position in which the first sealing means are positioned not to seal the outlet of a first capillary passage and the second sealing means are positioned to seal the outlet of a side passage.

Alternatively, respective first and second (and possibly further) sealing means may be provided for each of the capillary passage outlets, each operable for sealing the associated outlet or not. For instance, each sealing means may be located on a respective control element, e.g. axially movable towards and away from the associated outlet. As a further possibility, the sealing components may be located on a common control element, e.g. arranged for rotary or linear (lateral) motion, movable between a first position in which the first sealing means is in sealing relationship with the outlet of the first capillary passage, with the second sealing means not in sealing relationship with the outlet of the second capillary passage; and a second position in which the second sealing means is in sealing relationship with the outlet of a second capillary passage, and the first sealing means is not in sealing relationship with the outlet of a first capillary passage.

In an embodiment, it may be preferred to provide a pair of first and second sealing means on a common control element. Further pairs of first and second sealing means may be provided on the same control element as the first pair of first and second sealing means, or on different control elements.

In an embodiment, sealing means may operate in a binary manner between two positions, a position in which an outlet is sealed and a position in which an outlet is not sealed. In another embodiment, a sealing means may operate in a quantitative manner such that the sealing means may be operated to partially close an outlet, such that the rate of flow of the sample in a passage may be controlled depending upon the degree to which the outlet is opened or closed. For example, the sealing means may be operated to slide across the outlet, such that the rate of flow of the sample is slowed as the outlet is in a partially closed position. In an embodiment, the sealing means may adopt any one or more positions which partially close an outlet to alter the rate of flow in a passage. These embodiments may apply to both the first and second sealing means of the invention.

### Control Element

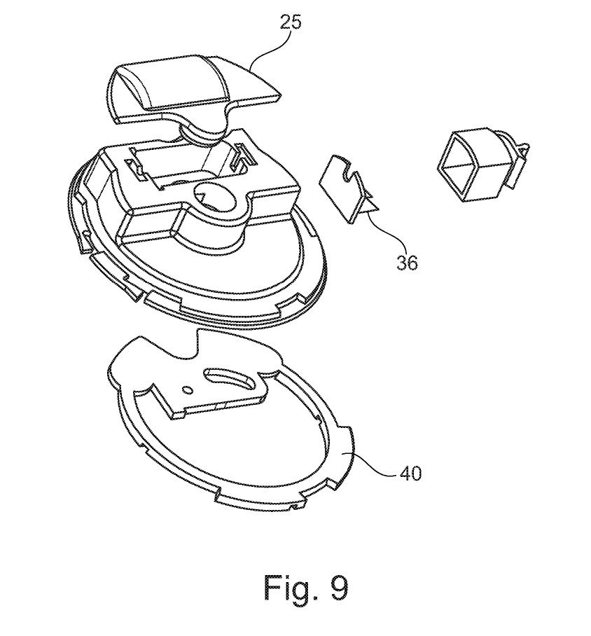

Sealing means (and additional sealing means if present) and/or a sealing component may be located on a control element, movable to cause operation of the sealing means. Each sealing means may be located on a respective control element. Preferably, all sealing means for a device are provided on, or operably linked to, a common control element. Preferably, a common control element may be a seal, as shown in FIG. 9.

A control element may be arranged for rotary movement or linear movement (axially, towards and away from the outlet, or laterally, in a sliding action).

Preferably, a control element conveniently surrounds a fluid application region.

A control element may be any suitable shape or size, preferably easily manipulated by the user. A control element may be of any suitable shape, preferably which allows it to move along or around a fluid application region. For example, it may be a rotatable element, for rotational movement about a pivot, or a formed for linear movement, e.g. a sliding motion along the location of outlets. Preferably, it desirably comprises a generally circular element, conveniently positioned for rotation with or around a pivot of the element. Other suitable shapes and forms of the control element and fluid application region are included within the scope of the invention. Grooves and elements may be provided on the control element and upper surface of the device to permit limited movement of the control element. A control element may be manually operable by a user, or automatically operable, for example prompted by one or more sensors associated with detection means in the device, or a timer.

A control element may comprise a well, or serve as a cap for a well. It may include a liquid inlet for passage of liquid to a fluid application region, and thus a first and/or second inlet. Preferably, the liquid inlet is in fluid communication with a fluid application region or well only when a control element is in selected positions, e.g. selected rotary or linear positions, as further described below.

Markings and/or stops are conveniently provided to indicate the various positions of the control element, to facilitate operation by a user. These may be provided preferably in the sample testing device.

Sealing means or sealing components may be carried on or form part of the control element, e.g. on the underside thereof. The sealing means or components may be constituted by elements, e.g. of soft material, e.g. a soft thermoplastic material such as an elastomer, standing proud of or forming part of the control element underside. In a preferred embodiment, a sealing component is a circular, planer element which sits adjacent to the underside of the control element. Alternatively, sealing means or a sealing component may be provided on a flange which extends outward from a side wall of a control element, preferably substantially perpendicular thereto. Sealing means may be feet, provided on a flange.

End stops are desirably provided to limit the movement of the control element.

Desirably, a control element is movable between

i) a first, inactive position in which a fluid (preferably sample) application region is shielded by the control element; a liquid inlet is not in fluid communication with the fluid application region or well; and the sealing means do not seal the outlet(s) of the capillary passage(s); and
ii) a second, sample application position, in which the fluid application region is exposed to a user and the sealing means do not seal the outlet(s) of the first capillary passage(s); and
iii) a third or further, fluid release position in which the control element is positioned to allow fluid to be released into the capillary passage, preferably via an inlet.

The inactive position may be used for storage or transit of the device, for example when provided as a complete device rather than as a kit of parts. It is the position adopted when the device is not in use. In the second position (sample application position) a sample application region is open, for example by operation of the control element to expose the sample application region to a user or to allow fluid communication between the sample application region and a sample well. In the second position (sample application position), the sealing means do not seal the outlet(s) of the capillary passage, so that sample is able to flow by capillary action along the capillary passage toward the outlet. In the third position (fluid release position) the control element is positioned to allow access to a fluid application region, for introduction of fluid such as buffer or substrate to the capillary passage. The position of the control element may be the same in the second and third positions, for example where the same application region and/or inlet is used for more than one buffer and/or substrate. Alternatively, where separate sample and fluid application regions are provided, the control element may be positioned to allow access to the different application regions sequentially in the second (sample application) and third or further (fluid release) positions. By “further” release positions is meant that the device can be maintained in the third fluid release position for the release into the passage of more than one fluid (e.g. additional buffers, substrate etc), or may be re-positioned into a fluid release position from a different position, preferably subsequent to the first fluid release step.

Where sample metering is provided for, a control element may be movable between:

i) an inactive position in which a fluid (preferably sample) application region is shielded by the control element; an inlet is not in fluid communication with the fluid (preferably sample) application region or well; and first sealing means do not seal an outlet of a capillary passage and second sealing means are positioned not to seal the outlet of any side passage; and
ii) a sample metering position in which the previously shielded fluid application region is exposed to a user and first sealing means are positioned to seal the outlet of the capillary passage and second sealing means are positioned not to seal an outlet of a side passage; and
iii) a reaction position in which the first sealing means do not seal the outlet of the first capillary passage(s), and the second sealing means seal the outlet of a side passage; and optionally
iv) a fluid release position in which in which the control element is positioned to allow fluid such as buffer or substrate to be released into the capillary passage, preferably via an inlet, preferably an inlet downstream of the sample inlet.

It is envisaged that for assays where a substrate is required for an enzyme or catalyst to act upon in order to produce a measurable signal, the substrate may be provided in a wash buffer or the substrate and wash buffer may be provided separately, for example via separate inlets. Preferably, wash buffer is provided in a second inlet, or upstream of substrate which may be provided via a third or further inlet. Buffer and/or substrate may be released into the capillary when the control element is in a fluid release position, either as a combined solution, or simultaneous release of separate solutions. Alternatively, a substrate may be provided separately to a wash buffer. Preferably, a substrate will be provided to the capillary passage after the wash buffer. In an embodiment, a control element may be movable between the positions as defined above.

Preferably, in a sample application position, a fluid application region or well is not exposed to the user. Preferably, in a fluid release position, a second inlet, or preferably a third, fourth or further inlet, is in fluid communication with a fluid application region and/or well.

Flow of the sample may be slowed, stopped and caused to resume flow by appropriate movement of the first sealing means, any number of times (one or more) during a single assay. This may be desirable in a multi-step assay, for example at a predetermined point to enable a reaction to occur before allowing the fluid to proceed to the next step. The invention can also be used to direct fluid, or a portion of fluid, along different capillary passages in a device.

Thus, an inactive position is used for storage or transit of the device, for example when provided as a complete device rather than as a kit of parts. It is the position adopted when the device is not in use. In a sample metering position, the device is prepared for use by opening the sample application region, for example by operation of the control element. A side passage outlet is open, and so sample applied to the sample application region in fluid communication with the first inlet flows along the capillary passage and into the side passage. A capillary passage outlet is closed to prevent flow of excess sample into the capillary passage. A first inlet and/or fluid application region may also be closed, to prevent backflow of sample toward the inlet. In a reaction position, a control element is positioned not to seal an outlet(s) of the capillary passage, allowing sample to flow along the reaction zone toward the capillary passage outlet. In the fluid release position, a fluid application region may be exposed to a user, or brought into contact with fluid dispensing means, for example by operation of a control element. In this position, fluid (e.g. buffer or substrate) may be applied to an inlet, preferably a second, third or further inlet. In this position, fluid may flow toward the capillary passage outlet. In an embodiment, a holding position may be provided prior to the fluid release position, in which fluid is brought into contact with a fluid application region or an inlet, preferably a second, third or further inlet, and the capillary passage outlet(s) remains sealed (for example by positioning of the control element). The capillary passage outlet can then be opened, such that the device is in the fluid release position and fluid can enter the capillary passage. Fluid (e.g. buffer) follows the test volume of sample along the capillary passage toward the capillary passage outlet in the assay. In an embodiment, the first sample inlet remains closed. The device may remain in the fluid release position for release of substrate, where appropriate, or may be moved to a holding position between fluid applications.

### Fluid Dispensing Means

In an embodiment, a fluid dispensing means (e.g. a fluid dispenser) may be provided. A fluid dispensing means may be an integral part of the sample testing device, or a separate element which optionally may be temporarily or permanently integrated with the device. The fluid dispensing means may be housed in a control element. A fluid dispensing means may comprise (i) a rupturable, sealed container of fluid to be dispensed, (ii) rupturing means for rupturing the container and releasing the contents; the container and/or rupturing means being arranged for relative movement between a first position in which the container is intact and a second position in which the container is ruptured. Where more than one container is provided, the additional containers may each independently be ruptured by the same rupturing means as the first container, or by additional rupturing means.

Fluid dispensing means may be used to provide buffer (e.g. chase buffer or wash buffer). They may also be used to provide substrate, where the signal is generated. Any buffer and substrate may be provided in separate containers, for release by the same or different dispensing means. Alternatively, they may be provided together, in the same container.

A rupturable, sealed container of fluid and/or rupturing means, e.g. in the form of projections in the vicinity of the fluid application region, may be movable with respect to each other for release of fluid. Operating means serve to move the container, rupturing means or both into a second position in which the container is ruptured. The operating means may be a plunger, carrying at one end either the container or rupturing means. Operating means may alternatively be arranged for rotary movement e.g. about a pivot, or linear movement (axially or laterally).

Preferably, at least a portion of a container wall is rupturable, e.g. being formed of rupturable foil such as a polyolefin film. A container may be made entirely of rupturable material e.g. being in the form of a capsule. As a further possibility, a container may mainly or partly comprise rigid material, e.g. a rigid plastics material, with a rupturable portion, such as a rupturable wall or base, e.g. of rupturable foil such as polyolefin film.

Any suitable rupturing means may be provided. Preferably, rupturing means conveniently comprise one or more projections, preferably having sharp tips. The projections are desirably tapered, and preferably have features to facilitate fluid release e.g. being of scalloped configuration. Desirably a plurality of projections are provided.

For a container, second rupturing means may similarly be provided, arranged to rupture an opposing portion of the container, to allow air to pass into the container. This aids flow of fluid out of the container. Second rupturing means may be provided as for the first rupturing means, provided they are arranged to rupture an opposing portion of the container.

Preferably, a rupturable container, at least when in a ruptured position, is in fluid communication with a well or inlet. Preferably, where a second inlet is provided, fluid dispensing means are arranged for fluid to flow from the container into the capillary passage via a second inlet, optionally via a well or application region.

In an embodiment where a control element is provided, this may carry fluid dispensing means. A control element may comprise a housing for a sealed container of fluid to be placed therein, and rupturing means. Preferably the housing is provided on the control element, as an integrated unit. The housing may comprise a lid, preferably hinged to a wall of the housing, for insertion of and access to the fluid dispensing means and rupturing means.

In an alternative embodiment, fluid dispensing means may be a separate element, which can be integrated with the sample testing device or a control element if provided, as described herein. Preferably, where this is the case, it may be provided as a kit of parts.

Alternatively, a fluid dispensing device may be composed of parts of the sample testing device and a control element. For example, rupturing means may be provided by the sample testing device (for example, as moulded upstanding projections), and a rupturable container and operating means may be provided by a control element.

In an embodiment, a single control element may be provided comprising sealing means (e.g. constituted by a sealing component), carrying means for a rupturable, sealed container of fluid (and optionally the container of fluid) and/or rupturing means and optionally operating means for bringing into contact a rupturable, sealed container and rupturing means. Such a control element preferably also defines a lid of a sample well or sample application region, by opening or closing the well or application region when moved between two positions.

In such an embodiment, movement of the control element to operate the sealing means may be combined with movement to open or close a well or fluid application region, and/or movement to rupture a container. Thus, for example, movement of a control element to operate the sealing means may also open or close a well and/or cause the container to be brought into contact with rupturing means. For example, in a preferred embodiment, a rotational movement of the control element may serve to open a well and seal the outlet of the capillary passage. A further rotational movement may drive operating means such that a container is brought into contact with rupturing means. In such an embodiment, a cam may be provided to operably link rotational movement of the control element with a linear movement of the operating means.

Alternatively, movement of the control element to operate sealing means may be independent of opening and closing of a well and/or from an operating means to bring the container into contact with the rupturing means. Thus, separate actions are required.

A container is preferably movable relative to the rupturing means, although other arrangements are possible, such as the rupturing means being movable relative to the container, or both being movable to come into contact.

In one preferred arrangement, a container is arranged for downwards movement, to be brought into contact with rupturing means. In this embodiment, rupturing means are preferably provided on a control element, and preferably are in fluid communication with a sample well or fluid application region. Rupturing means may comprise projections, and the container is impaled onto upstanding projections. In another preferred embodiment, the container is arranged for impaling on projections and being pierced by spikes. In an alternative embodiment, rupturing means may be provided adjacent to the fluid dispensing means, and arranged for axial movement, to rupture the dispensing means. Rupturing means may be provided on an inner side wall of the housing.

Preferably, a container or rupturing means are movable within a control element between the first and second positions, e.g. operable from the exterior of the control element by simple application of force, e.g. manually by a user or in automated manner. The relative movement between rupturing means and a container may be axial or linear (i.e. the movement of the operating means may be linear or axial). Activation brings rupturing means and a container into contact, thus releasing fluid from a container. Preferably, the same action brings second rupturing means into contact with a container, to allow air to pass into the container. Thus, preferably, fluid passes passively from the container.

The fluid dispensing means is conveniently used to dispense fluid to a fluid receptacle, e.g. for reaction therein, or to the inlet of a fluid flow passage.

This embodiment of the device of the invention is conveniently used for supplying a known volume of reagent, e.g. a buffer or substrate, to the system. This enables the assay to be carried out using a smaller quantity of sample than would otherwise be required.

The embodiment can enable fluid to be dispensed reliably in known quantities, determined by the container contents, even small volumes such as 1000 microlitres or less, 500 microlitres or even less.

In an embodiment, the fluid dispensing means may comprise a further container for substrate solution. In an embodiment, the substrate solution container is ruptured independently of the buffer container. Preferably, release of the substrate solution is controlled by a control element, preferably the same control element as that controlling the chase buffer container. Separate fluid dispensing means may be provided for a container of substrate solution. Alternatively, a container of substrate solution may be provided in, and released by, the same fluid dispensing means as described above in relation to buffer. In the latter case, the fluid dispensing means are preferably arranged to allow for release of substrate solution at the same time as buffer, or at a set period of time after the release of buffer.

### Reagent Zone

A capillary passage of the sample testing device may comprise reagent deposited therein, preferably at one or more discrete locations to define a zone, for example a reagent zone. Alternatively, reagent may be provided to a reagent zone during the assay, for example prior to sample introduction into a capillary passage. In such an embodiment, the reagents are wet (i.e. not dried in the passage and requiring reconstitution), although dried reagents are also included. Any suitable methods may be used for provision of reagent in a capillary passage. Reagents may include, for example, agglutination reagents, binding members, substrate, and labels (for example signal linked binding members or signal linked analyte analogues). Other reagents include buffers, and any other assay components. A reagent zone may be positioned between an inlet and capture zone and may comprise a signal linked binding member. Preferably, the binding member is an enzyme linked binding member. Provision of a specific binding member in a reagent zone upstream of a capture zone allows time for binding of analyte to the binding member in the reaction zone, thus increasing the sensitivity of the assay. A reagent zone may comprise a binding member of the capture zone (analyte analogue or analyte specific binding member) which is later immobilised in the capture zone, and a signal linked binding member. Such an embodiment increases the time available for reaction between analyte, capture binding member and signal linked binding member.

Where a side passage is provided for metering, a reagent zone is preferably positioned downstream thereof.

Other sample treatment reagents (for example, an anticoagulant) may be provided in or adjacent to a reagent zone, preferably upstream of any junction with a side passage.

Reagents may be dried into the capillary passage in a reconstitutable form. Any suitable method for depositing the reagents (e.g. addition of a defined volume of fluid via a pipette, microdroplets, ink-jet printing, etc.) or drying (e.g. heating, desiccation, vacuum drying, lyophilisation, etc) can be used. Reagents may be reconstituted by passage of the sample through said zone

Reagents may be dried onto a separate element which is then inserted into the capillary, thus simplifying manufacture. Alternatively, the reagents can be dried into a bead or pellet which is inserted into an area of the device during manufacture.

Any suitable reagent formulation can be used. Preferably, it will suitable for long-term stability of the reagents, and is rapidly reconstitutable by sample. Formulations containing sugars have been found to be especially suitable. Other formulations will be known to persons skilled in the art.

Typically, a signal linked binding member will be provided in excess, such that if analyte is present, all can bind to signal linked binding member.

### Reaction Zone

A reaction zone is defined by the capillary length between the reagent zone and capture zone. Within this length, sample and reagent interact within the capillary lumen during flow downstream toward the capture zone. In an embodiment, any analyte present in the sample may bind to signal-linked binding member provided in the reagent zone, and to the capture binding member if it is provided within the reagent zone.

The reaction time can be pre-determined by providing a capillary passage lumen of the reaction zone of known dimensions and shape, taking into account factors such as migration speed. Thus, it will preferably take the sample and reagents a finite time to pass from the reagent zone to the capture zone. The advantage is that timing of the reaction requires no external influence or operator intervention, unlike conventional heterogeneous assays for example ELISA assays.

### Capture Zone

A capillary passage of the sample testing device comprises a capture zone which serves to capture a population of signal linked binding member, to provide a “bound” fraction and a “free” fraction of the signal linked binding member. The distribution between “bound” and “free” fractions of the signal linked binding member is dependent upon the concentration of analyte in the sample. The measurement of the bound and/or free fraction provides an indication of the amount of analyte in the sample. Two or more (three, four or five or more) capture zones may be incorporated into the device to measure both bound and free fractions of the signal linked binding member. Where more than one capture zone is provided, the terms “bound” and “free” are used in reference to the first capture zone downstream of the reaction zone.

A capture zone effects the separation by retaining one of the fractions in the zone, such that when wash buffer is added to the capillary passage, the fraction which is not retained passes downstream, away from the capture zone.

Any suitable means may be used to capture a bound or free fraction of signal-linked binding member in a first capture zone, many examples of which will be known to persons skilled in the art including physical trapping (for example based on size) or chemical or biological trapping (for example based upon reaction with an immobilised reagent). The latter includes, for example, immunological trapping.

Where biological trapping is used, one member of a binding pair (e.g. analyte analogue or an analyte binding member) may be directly or indirectly immobilised in the capture zone. The other member of the binding pair will be the analyte or analyte analogue. In an embodiment, a binding member for analyte may be immobilised in the capture zone.

Indirect immobilisation may utilise a coupling mechanism, for example a ligand receptor pair, to immobilise a binding member in the capture zone. One member of a ligand-receptor pair may be conjugated to the binding member to be immobilised (e.g. analyte analogue or analyte specific binding member), and the other member of the ligand receptor pair may be immobilised in the capture zone. Binding of the ligand and receptor thus causes immobilisation of the binding member (e.g. analyte analogue or analyte specific binding member) in the capture zone. Examples of ligand receptor pairs include biotin and avidin or streptavidin. Thus, for example, a biotinylated binding member (analyte binding member or analyte analogue), may be immobilised in the capture zone by providing streptavidin therein, e.g. coated onto the capture zone, for example on the fins. As the reaction mixture passes through the capture zone, biotinylated binding member may be captured by streptavidin. Any unbound reagent may be washed downstream by the subsequent addition of wash buffer.

It is envisaged that a binding member may be immobilised in the capture zone prior to the assay (for example by indirect or direct coupling, as described above). Alternatively, a binding member may become immobilised in the capture zone during the assay. In such an embodiment, the binding member of the capture zone (analyte analogue or analyte specific binding member) may be provided to the assay upstream of the capture zone, for example in the reagent zone or in a buffer, released with or after sample, into the capillary passage. Indirect coupling may be used to immobilise the binding member in the capture zone. For example, the binding member to be immobilised may be conjugated to a first member of a ligand receptor pair, the second member being provided in the capture zone. During the assay, as fluid enters the capture zone, any binding member will become bound by the second member of the ligand receptor pair, and become immobilised. In a preferred embodiment, the ligand receptor pair is biotin-avidin or streptavidin. In a preferred embodiment, avidin or streptavidin is provided in the capture zone, and biotin is conjugated to the binding member to be captured. In this way, capture within the capture zone relies upon ligand-receptor binding within the capture zone.

Where a binding member or member of a ligand-receptor pair is immobilised in a capture zone, this may be achieved using any suitable means, including covalent or non-covalent means known in the art. A preferred option is non-covalent adsorption of reagent to hydrophobic regions on a capillary passage.

Alternatively, size based filtration may be used as a capture means. Suitable reagents may be provided which create a difference in size between a fraction to be retained in the capture zone and a fraction to be washed downstream. For example, agglutination reagents may be provided to cause agglutination in the presence of analyte, such that an agglutinate may be trapped by filtration in a capture zone. Suitable agglutination reagents will be known to persons skilled in the art, and may include a bead or soluble hub, for example a macromolecule, preferably a linear macromolecule, such as polysaccharides, including dextran, preferably aminodextran, agarose, microcrystalline cellulose, or starch.

Alternatively, a member of a binding pair may be attached to a particle, such as a bead, whilst another is signal linked. In this embodiment, the particle becomes trapped by the filter, together with the fraction of signal linked binding member which is analyte bound, whereas the non-analyte bound fraction is washed downstream, thus effecting separation of the bound and free fractions. Suitable filters may be of any suitable form which have an effective pore size which will trap a fraction to be captured (e.g. comprising an agglutinate or particle). Examples include filter paper, nitrocellulose, sintered frits, and other filters known to persons skilled in the art. Features provided to increase surface area, as described herein, may also serve as a filter, for example microstructures as described herein, for example closely-spaced micropillars.

A capture zone may be any suitable size and shape. It may be have similar dimensions and shape to the rest of the capillary passage, or may have a different size and shape thereto. Preferably, the capture zone is configured to maximise capture of a fraction, for example by maximising the surface area of the capture zone. Preferably, a capture zone is a widened portion of a capillary passage. Thus, it may not be a capillary passage, but may represent an interruption thereto. Preferably, it is shaped such that flow of liquid is not impeded. A suitable shape for the capture zone may be oval, diamond, trapezoid, triangular, rectangular or any other. In an embodiment, a broadened area of the capillary has essentially parallel sides with a width of 1-20 mm, ideally 3-10 mm, most preferably 5 mm. To ensure continuity of fluid flow there may be a tapered region leading into and out of a capture zone linking it to the main capillary passage, for example as described herein.

A widened/tapered portion may comprise microstructures, as described herein, to aid flow between a capillary passage and capture zone.

A capture zone may incorporate microstructures, as described herein (e.g. pillars, cones, roughened areas, fins, appendages, etc) to increase its surface area. This provides a greater surface area for immobilisation of a bound or free fraction. This serves to increase the efficiency of capture. The design of the features preferably is such that they do not significantly impede flow of liquid, for example the wash process to separate bound and free fractions.

A capture zone may comprise a plurality of fins which increase the surface area of the capture zone to maximise capture of signal linked binding member. In an embodiment, a fin is a thin component or appendage, attached to a larger body (e.g. a base), to increase surface area of the body. Within the parameters defined above, the fins may be any shape or size, e.g. rectangular, square, tapered etc. Fins of different shapes and assizes may be included in a single measurement zone. The nature of the fins and capture zone may be as described herein.

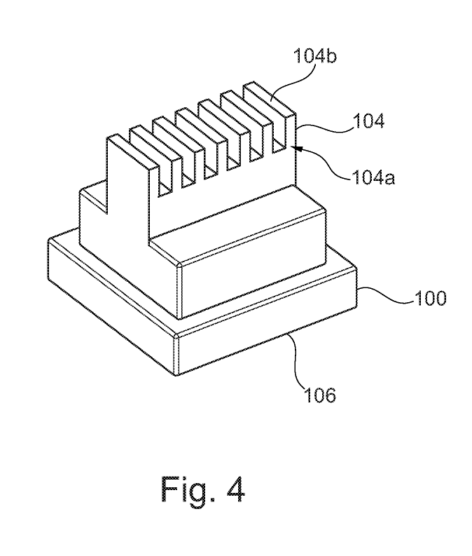

The fins may be produced as a separate item which can be inserted into a capillary passage. This allows for separate production of the capillary device and any treatments to be performed independently of the capture zone, greatly simplifying manufacture (see FIG. 4).

A first capture zone is preferably provided centrally in the device, between a fluid application region at one end and a sump at the opposite end. Preferably, where the capture zone comprises a capillary passage of serpentine configuration, a capillary passage enters a capture zone from one side of the device, and leave the capture zone on the opposite side of the device.

### Signal Measurement Zone

A signal measurement zone (SMZ) will be configured to enable detection and measurement of a signal, for example signal generated by reaction of a substrate and catalyst or enzyme. Typically, this may be an optical measurement, and the signal measurement zone will then be designed to provide a light path across it.

In a preferred embodiment, a signal measurement zone is combined with a capture zone. A combined zone preferably comprises means for directing an optical pathway across or through the combined capture and signal measurement zone. In a preferred embodiment, a combined capture and signal measurement zone includes the plurality of elongate fins projecting substantially perpendicularly from a base, where each elongate fin has a length that is substantially parallel to the base, the elongate fins being arranged so that:
the lengths of the plurality of elongate fins are substantially parallel to one another;
the plurality of elongate fins are aligned along a line that is substantially perpendicular to the lengths of the fins; and
the lengths of the plurality of elongate fins are substantially perpendicular to said optical pathway;
said plurality of elongate fins permitting optical transmission therethrough along said optical pathway and defining a plurality of fluidic channels therebetween along the base for receiving fluid from said capillary pathway.

One or more of the capture zones provided in the capillary passage may independently be a combined capture and signal measurement zone, preferably as described herein. Such a design offers significant benefits over existing designs, including:
provision of a large surface area for capture of bound signal fraction
minimal resistance to flow for efficient washing
a long optical pathway to increase sensitivity.
a reduced mean-free path for reactants, to increase the rate of catalyst or enzyme and substrate reaction

“Fins” act as a capture surface to bind signal linked binding member, which is retained on the fins during washing, thus effecting separation of bound and free fractions. Signal may be measured by directing light across the signal measurement zone and through the fins. The fins may extend parallel to the sides of the signal measurement zone, thus reducing bending of light of the optical pathway. Preferably, the fins are also perpendicular to the direction of the optical system, to minimise interference in the measurement process. Herein, a fin is a thin component or appendage, attached to a larger body (e.g. a base), to increase surface area of the body. Within the parameters defined above, the fins may be any shape or size, e.g. rectangular, square, tapered etc. Fins of different shapes and assizes may be included in a single measurement zone.

The fins may be produced as a separate item which can be inserted into the device in order to be fluidly connected to the capillary passage i.e. the capillary passage and the fluidic channels are in fluid communication. Thus, it may be inserted into the capillary passage, or may be adjoined to the capillary passage such that the aforementioned fluid communication is possible (e.g. looped regions adjoin fluidic channels). This allows for separate production of the capillary device and any treatments to be performed independently of the signal measurement zone, greatly simplifying manufacture (see FIG. 4).

The device may further comprise end regions, which when the fins are aligned with the looped regions to form fluidic channels, an end region sits next to a fin. An end post may serve to further define the shape and form of the fluidic channel defined by the fins and looped regions. For example, end posts may be curved, corresponding to the shape of the inside of a looped region, such that the fluidic channel defined by the looped region, end region and fin has a uniform width around each loop. The distance between the fins defines the width of a fluidic channel, and is therefore preferably the same as the width of a capillary passage. Preferably, a plurality of fins are evenly spaced, such that a serpentine capillary passage defined by the fins and looped regions have an even width through a capture and/or signal measurement zone. The distance between two or more fins Thus, preferably, a fin will have the same width as the distance between

Thus, prior to insertion of the insert, the capillary passage may comprise an open space or cavity, into which the insert is to be placed. The open space may comprise looped regions on one or both sides thereof, preferably along the sides of the open space parallel to the optical pathway. A looped region may be a semi-circular, or where an end region is provided, the looped region defined by the loop and end portion may be C-shaped. The lopped regions may be positioned alternately on upstream and downstream sides of the open region, and where end regions are provided, these are preferably provided within a loop and so may also be provided alternately on upstream and downstream sides of the open region. Each fin preferably sits perpendicular to the optical pathway, end on into a looped region. Where an end post is provided, an end of a fin preferably abuts an end region within a looped region.

A capture and/or signal measurement zone may comprise 2, 3, 4, 5, 6, 7, 8, 9, or 10 or more fins, or may comprise 2, 3, 4, 5, 6, 7, 8, 9, or 10 or more fluidic channels defined by the fins and/or looped regions. The capture and/or signal measurement zone may be configured to allow simultaneous or sequential filling of the fluidic channels.

All or part of a measurement system may be provided in a co-planar location to the optical pathway. This allows a measurement system (e.g. light source and light detector) to be surface-mounted on the device yet still direct the optical pathway through the signal measurement zone. Suitable light directing means may be provided for re-directing the optical pathway as necessary. For example, a measurement system comprising a light source and a light detector may be provided in a co-planar location to the optical pathway, and a pair of prism shaped mirrors or other light directing means may be provided to turn the light into the direction of the optical pathway through the fins. Preferably, the light directing means may be capable of turning light through 90°. Alternatively, the measurement system may be provided in the same axis as the optical pathway through the fins. In an embodiment, a measurement system is provided on a planar element, separable from the device.

A common measurement system may be provided for one or more signal measurement zones of one or more capillary passages.

In an embodiment, any optical components of the device may be transparent. For example, they may be transparent plastic, for example polycarbonate. In a preferred embodiment, the remainder of the sample testing device, or those regions surrounding the light path may be opaque (e.g. polycarbonate containing a black dye) to absorb any light which is not substantially perpendicular to the fins.

### Measurement System

Any suitable measurement system compatible with the signal can be provided. This may be separate to, or integrated with the device. A measurement system may measure the signal of the bound fraction of the capture zone, or the free fraction (e.g. captured in a second capture zone), or both. Two or more measurement systems may be provided in relation to a single device.

Any suitable method of measuring signal may be employed, depending upon the nature of the signal. Where the signal can be detected optically, measurement of light absorption or transmittance may be performed. In such a case, the measurement system may comprise a light source and light detector. A preferred method is to measure the attenuation due to absorption of any electromagnetic radiation, or more specifically of an optical wavelength. Any suitable wavelength may be used, for example between 350 nm to 1000 nm i.e. it would also include the use of infra-red or ultraviolet radiation beyond the optical range.

In an embodiment, either the relative change in attenuation of any single wavelength may be measured, and/or the relative change in absorption or transmittance between different wavelengths over the course of the test may be measured. The latter is preferred. For example, if using a substrate which generates a blue colour in the presence of enzyme it is possible to measure a significant change in attenuation of red light at 630 nm, which may be referenced to blue light at 470 nm which will experience little change in attenuation during the test. Similarly, it is possible to measure green light at 530 nm and observe that the relative change in the attenuation of all wavelengths were in the correct proportions to each other. Typically 3 wavelengths may be measured. The choice of wavelengths depends on the optical transmission/absorption spectra of the biochemical reagents and how that changes over the period of the reactions. Throughout the present application, references to optical radiation and similar terms are in relation to any electromagnetic radiation and are not limited to any particular wavelength range.

The change in optical attenuation is proportional to the amount of analyte present.

Any source of light/radiation and light detector may be used. Examples include an LED light source and/or a silicon photodiode.

In a preferred embodiment, a light source is provided to direct a light path through the signal measurement zone. A photodetector may be provided on the other side of the zone. Any signal present (e.g. generated by reaction of substrate with enzyme-antibody) will absorb light such that the light reaching the photodetector is attenuated. The degree of attenuation will depend in part on the amount of enzyme present, and thus the analyte concentration of the sample being measured. The sensitivity of the system can be enhanced by increasing time for the enzyme reaction to occur (the longer the duration, the greater the signal) and the light pathlength (the longer the greater the signal).

### Signal Processing and Data Reduction Means

A sample testing device of the process invention may incorporate a mechanism to convert measured signal to a readable output of analyte concentration. The output may be provided in any suitable format, for example for the signal measurement (e.g. absorbance) at a pre-determined time; the rate of reaction; or signal vs time. Preferably, the output is adjusted to account for any background signal which may be measured prior to, or during the assay.

The relative change in optical transmission at the wavelength of maximum expected change ₘₐₓ, and at any other wavelengths of interest is compared with the relative change in transmission at the wavelength of minimum expected change, ₘᵢₙ.

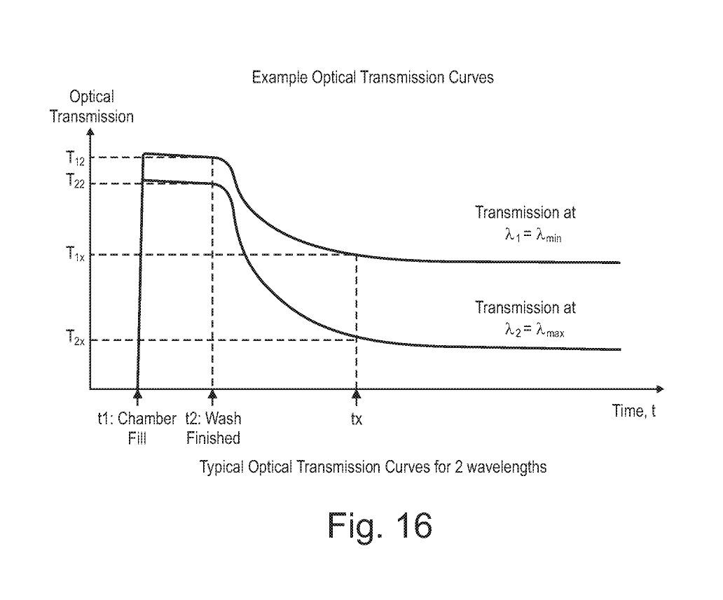

From this, the rate of change of the substrate colour can be determined. This will be a measure of the analyte concentration, as illustrated by FIG. 16.

The relative change in optical transmission at time tx relative to that at time t2 would be:

$${T}_{rel} = \frac{{T}_{22} - {T}_{2 x}}{{T}_{12} - {T}_{1 x}}$$

This is just one possibility for a relative measure of change in transmission.

The rate of change of colour may be established by—
i) measuring Tᵣₑₗ at a fixed time tx. Hence the average rate of change would be obtained between t1 and tx.
ii) measuring the time taken (tx−t2) for a fixed Tᵣₑₗ to occur.
iii) measuring change or the rate of change of Tᵣₑₗ by sampling around a fixed point in time, tx.

A “dose response curve” (DRC) would be used to infer an analyte concentration based on the rate of change of Optical Transmission. This DRC is obtained by running large numbers of test capillary chips with known analyte concentrations and observing rates of change of transmission. Any suitable DRC may be used, for example a 4 or 5 parameter logistic function, spline function etc.

Signal processing means convert measured signal to analyte concentration. The signal processing means are capable of converting the results from the signal measurement to a readable output on a display. Signal processing means may include a timer which is activated at an appropriate point in the assay. Thus, the signal processing means communicate with the detection means, converting the measured result to a digital or other format output. This output is then used to calculate the concentration of analyte in the sample using, for example, a dose-response algorithm, look-up tables, etc. in the on-board microprocessor. Additional algorithms to compensate for environmental influences (e.g. temperature) and/or reagent degradation, substrate deterioration, etc. may optionally be incorporated.

The calculated result can them be transmitted to a display device, which will present the signal is a readable format. This may be a yes/no type result, in the form of words or signs, or may be a quantitative result providing a value which is indicative of the amount of analyte present. In an embodiment, the device may take the form of “write-once” electro-chemical display or digital data transmission for record keeping or remote assessments as described in PCT application No. PCT/GB2005/004166, incorporated herein by reference.

Alternatively a result decision and raw data may be transmitted by wired, wireless far field or wireless near field communication techniques to a receiving “reader” docking device. A reader would be capable of relaying the information to a computer or through a computer network to a remote computer or to a hand held computing device (e.g. smart phone or tablet computer). Such a computing device could provide electronic storage and also permit more detailed analysis such as but not limited to trend analysis. The results could also be made available to a remote clinician.

### Detection Region

In a preferred embodiment, a capillary passage may comprise detection means for detecting presence or absence of sample or fluid. This enables the operator to confirm that fluid has entered and flowed to correct position(s) in the device during an assay. Such means may be used to communicate to the user that further operation of the device (e.g. sealing or not sealing an outlet) is necessary, and/or to monitor flow for the purpose of obtaining assay results or as a control mechanism to confirm that the device is performing satisfactorily. A side passage may comprise means for detecting the presence or absence of sample, preferably to confirm that sample has entered the side passage, and therefore the test volume is present in the main capillary passage (i.e. the volume is not short or insufficient). Suitable detection means for use in the invention may include, in a simple form, for example a viewing window, or other means such as optical, electrical, electronic or elctro-optic means. A series of detection means (i.e. two, three, four or more) may be provided in a capillary passage. A detection means is preferably operably linked to a signal processor of the device, to enable signals to be provided to the user for operation of the device. A detection means may be operably linked to a control element, for operation of a sealing means of the device.

A detection region may be provided at the end of the fluid sump to indicate when washing is complete, and/or to indicate when measurement of signal may be commenced. A detection region may also be provided at the intersection of a capillary passage and any associated side passage to indicate when sample metering is complete. Further detections regions may also be provided where desired.

Two or more detection means and/or detection regions may be provided in any capillary passage.

### Fluid

Herein, fluid is used to refer to non-sample fluids which are used in the assay, for example buffer or substrate.

A buffer may be used to assist movement of the sample in the passage, although the fluid may be any fluid required for performance of the assay. Herein, the buffer may be referred to as a wash buffer or a chase buffer. Any suitable buffer may be used, for example, a solution of phosphate buffered saline, Tris saline, etc. The use of a buffer enables the reaction to be carried out with a smaller volume of sample than is required to flow around the entire capillary system to determine a test result.

In an embodiment, a wash buffer is used, which serves to wash unbound reagent and material from the capture zone downstream toward the fluid sump and which does not react with any reagents.

The wash buffer may incorporate a surfactant (e.g. Tween 20) to assist washing away of unbound components.

The buffer may comprise substrate where the assay employs an enzyme or catalyst-substrate based signal system. Alternatively, a substrate may be provided separately.

Herein, the terms wash buffer and chase buffer may be used interchangeably.

### Wash Zone

A wash zone is the region of capillary which extends from the capture zone to the outlet or fluid sump. A wash zone is configured in terms of dimension to hold a volume sufficient for washing of the capture zone to effect separation of bound and free fractions. In embodiments where additional capture zones are provided to capture a free fraction, these may be provided in the wash zone. A wash zone may include detection means, as described above, for example to determine when washing is complete.

### Fluid Sump

A fluid sump may be provided, to minimise the length of capillary required to accommodate the volume of wash buffer required. A fluid sump may be provided in the wash zone, or downstream of the wash zone. A fluid sump stores the sample and any buffers and liquids which have flowed downstream from the combined capture and signal measurement.

A fluid sump may be a cavity of suitable size and shape, for example a circular cavity, or may be n elongated or widened portion of capillary (e.g. a long capillary section, for example in the form of a spiral), a split capillary, or may be a reservoir (for example a void, for example provided between flat sheets preferably of the device, and preferably which is configured to enable capillary flow but which does not comprise a capillary passage lumen as defined herein) fluidly connected to the capillary passage and an outlet of the capillary passage. The size and shape of the fluid sump is designed to enable continuous fluid flow through the capillary passage, and therefore preferably is capable storing sufficient volume to hold sample and wash buffer. Preferably, a sump comprises a capillary which branches into two or more capillaries, wherein the two or more branches form a spiral. Preferably, the sump is provided at the opposite end of the device to the fluid application region.

Preferably, the end of the device is curved to accommodate the shape of the spiral fluid sump. A pad of absorbent material may be included as a means of enhancing the absorbance and storage characteristics of the fluid sump.

A fluid sump is fluidly connected to an outlet such that fluid is drawn into the fluid sump by capillary action when the outlet is open.

A fluid sump may comprise an outlet. In an embodiment, an outlet may adjoin and/or lie below a fluid sump, for example as shown in FIG. 17.

A fluid sump may comprise an absorbent pad. A pad may be shaped to fit tightly within the sump, as shown in FIG. 17B.

The combined volume of the fluid sump and capillary downstream of the capture zone may define the wash volume of the system.

### Environmental Monitoring & Control

The flow of fluid in the sample testing device, and the biochemical reactions may be influenced by temperature. A sample testing device of the invention may comprise means for controlling and/or monitoring the temperature of the device (e.g. to heat or to compensate for environmental temperature and/or other environmental conditions). Such means will generally be known to persons skilled in the art, and may include electronic means. The measurement of temperature may be achieved with standard temperature transducers such as thermocouples and negative temperature coefficient (NTC) resistive devices.

### Semi-Integrated Device

Any heat, electrical power and optical sources and sensors may be mounted on the sample testing device or be provided separately thereto, for example on a separate docking/reader station. Near Field Communications (NFC) may be used to wirelessly retrieve data from the test device to the docking station. Wired connections are also possible.

### Sample

A sample may be any liquid or fluid sample. Preferred samples for assay using the present invention are blood (whole blood or serum/plasma), saliva, and urine. Herein, the terms liquid and fluid may be used interchangeably.

Non-biological samples may also be used.

### Analyte

Analyte may be any moiety, preferably one which is capable of being bound by a binding partner. A non-limiting selection of analytes include nucleic acid, antigen, antibody, oligonucleotide, hormone, hapten, hormone receptor, vitamin, steroid, metabolite, aptamer, sugar, peptide, polypeptide, protein, glycoprotein, organism (such as fungus, bacteria, viruses, protozoa and multicellular parasites), therapeutic or non-therapeutic drugs, or any combination or fragment thereof. Preferably, the analyte may be an immunologically active protein or polypeptide, such as an antigenic polypeptide or protein. Most preferred analytes for detection by the present invention include hCG, LH, FSH, and antibodies to HIV. As will be clear to those of skill in the art, antibodies are particularly important analytes where evidence of an immune reaction is being measured. Accurate measurement of serum titres of particular antibodies is therefore an important aspect of the invention. In such assays, it will be understood that the analyte-binding reagent used is usually an antigen to which the antibodies being measured specifically bind.

An epitope is a single site upon the analyte to which a binding partner is capable of binding.

### Immobilisation

Where a binding partner or ligand-receptor pair is immobilised, for example on a particle or on a surface of the device, any suitable manner of attachment may be used, either covalent or non-covalent. Suitable methods include covalent links such as for example, chemical coupling, or by non-covalent links such as antibody-antigen interactions, biotin-streptavidin, protein-protein interactions, protein G or protein A interactions, or passive adsorption. Preferably, the covalent link is formed between an amino acid, typically an amino acid side chain, such as an amino, sulphydryl, carboxyl, phenolic or other heteroaromatic or aromatic side chain.

To achieve non-covalent binding as described above, a binding member may be provided as a conjugate, wherein a binding member is coupled to a further binding partner capable of binding the particle or surface. An embodiment is described above, where a ligand-receptor pair is employed. This binding is preferably via sites distal to their analyte binding sites such that any interference with analyte binding is reduced or avoided. Where the binding partners are antibodies, such sites may be the tails of the binding partners such that coupling occurs in a tail-tail manner. The coupling may be covalent, for example via amino, sulphydryl carboxyl, phenolic or other heteroaromatic or aromatic side groups of an amino acid of the binding partner, or preferably via a thiol group. Alternatively, the coupling may be non-covalent, as described above.

### Binding Member

A binding member of the present invention may be any substance which is capable of binding a predetermined target (such as an analyte or analyte analogue) and preferably which has a preferential affinity for said predetermined target (i.e. is specific for that target). Binding members therefore include monoclonal or polyclonal antibodies, antigens, proteins including enzymes or other binding proteins, receptors, aptamers, oligonucleotides, analogues, sugars, and fragments thereof. The binding members may be selected from the above based upon the nature of analyte. Preferably, a binding member may be an antibody, such as a known immunoglobulin, e.g., IgG, IgM, and the like, or monovalent and divalent antibody fragments of IgG, conventionally known as Fab and Fab′, and (Fab′)₂, respectively, or a fragment thereof. Preferably, the antibody will commonly be a divalent antibody fragment [(Fab′)₂] or, more preferably, a monovalent antibody fragment (Fab or Fab′).

Whilst it is preferred that the binding members bind their targets directly, this is not strictly necessary, and the binding may take place via an intermediate, such as an analyte binding molecule. The intermediate might be naturally present in a sample, or may be separately provided. These include receptors, antibodies, antigens, binding molecules, hormone receptors, oligonucleotides, sugars, or aptamers, as described above in relation to the binding partners etc.

### Fractions

Herein, the terms “bound” fraction and “free” fraction are used, and describe the condition of retention by the first capture zone of a capillary passage. This capture zone may be referred to as the first capture zone. Binding in a first capture zone may be wholly or partly determined by the presence or concentration of analyte in the sample. Thus, the term “bound fraction” refers herein to a population of signal-linked binding member which becomes retained by a first capture zone. Thus, conversely, the “free” fraction is the population of signal linked binding member which is not retained by a first capture zone, during flow of sample therethrough. In those embodiments where a second or further capture zone is provided to capture the free fraction or a control marker, this fraction is still referred to as free because it has not been captured by a first capture zone downstream of the reaction zone. In embodiments, a second or further capture zone may be provided for capture and measurement of the free fraction.

The present invention is applicable to a wide variety of assay formats, including (but not limited to):
A. A 2-site assay format utilising a pair of binding members, one member of which is or becomes immobilised in the capture zone. The other member of the pair is the signal-linked binding member of the reagent zone, which reacts with any analyte in the sample to form a bound signal-linked binding member. The other of the pair of binding members is or becomes immobilised in a combined capture and signal measurement capture zone, where it binds to analyte (already bound to the signal-linked binding member), thus capturing the bound signal-linked binding member in the combined capture and signal measurement capture zone such that the bound fraction of signal-linked binding member is proportional to analyte concentration. Any unbound signal linked binding member may be captured and measured in a second or further capture zone.
B. A competitive assay format, utilising a binding member which is or becomes immobilised in the combined capture and signal measurement capture zone. Analyte competes with signal-linked analyte analogue for a limited number of binding sites on the immobilised binding member. The bound fraction of signal-linked analogue is thus inversely related to analyte concentration. Unbound signal linked analogue may be captured and measured in a second or further capture zone.
C. A 1-site assay format, which utilises an analyte-analogue which is or becomes immobilised in the combined capture and signal measurement capture zone. Signal-linked binding member of the reagent zone will react and bind to analyte; any signal-linked binding member which is not bound to analyte will become bound by analyte-analogue which is or becomes immobilised in the combined capture and signal measurement capture zone.

A wide variety of other assay formats are also well known, including assays for specific antibodies. The “free fraction” is the population of signal-linked reagent which is not so bound within the combined capture and signal measurement capture zone. This can be captured and measured in a second or further capture zone.

By measuring the amount of signal linked binding member captured, or free binding member, or both separately (e.g. by signal measurement) the amount of analyte in the sample can be determined.

### Display Means

The display means acts as an interface between the device and user and provides a readout of result obtained from the Signal Processing Means. Preferably, the means incorporates the technology described in PCT/GB2005/004166 which provides a permanent or semi-permanent readout of results, rather than systems such as LCD's which can only display information so long as there is battery power to maintain the display.

### Timer

Optionally, a timer is associated with a device of the invention. It may be integrated within the device, or provided separately thereto. The timer may be used to indicate the time for operating sealing means or a control element.

### Power Source.

A power source may be incorporated in the device to provide energy for features such as signal measurement, data reduction means, timer, optional heater and display means. A suitable power source may be a battery pack on board the device (permanently or temporarily integrated). Coin cells may be used. Where a battery is used, it may be isolated during storage (to prolong battery life) and automatically connected to the circuit when the device is operated. Alternatively, a power source may remain connected to the device during storage, for example to monitor temperature.

Alternatively, power may be supplied from a reader device that wirelessly provides power by near field magnetic induction.

### Power Switch

A power switch may be included to minimise on-time and hence minimise battery drain on the device.

### Kit

In a third aspect of the invention, the present invention provides a kit comprising
i) a sample testing device comprising a capillary passage having a lumen;
ii) a combined capture and signal measurement zone including a plurality of elongate fins projecting substantially perpendicularly from a base, where each elongate fin has a length that is substantially parallel to the base, the elongate fins being arranged so that:
   the lengths of the plurality of elongate fins are substantially parallel to one another;
   the plurality of elongate fins are aligned along a line that is substantially perpendicular to the lengths of the fins; and
   the lengths of the plurality of elongate fins are substantially perpendicular to said optical pathway;
   said plurality of elongate fins permitting optical transmission therethrough along said optical pathway and defining a plurality of fluidic channels therebetween along the base for receiving fluid from said capillary pathway.

In an embodiment, the capillary passage may comprise a widened portion into which combined capture and signal measurement zone is inserted, preferably immediately upstream and/or downstream thereof. Alternatively, the capillary passage does not form a continuous fluid path and instead includes a series of disjointed looped portions. When the combined capture and signal measurement zone is inserted, the looped portions of the capillary passage and the fluidic channels between adjacent fins together form a single fluidic channel.

Thus, a capillary passage of a sample testing device of a kit may be disjointed, comprising two or more separate portions which upon insertion of the combined capture and signal measurement zone, form a single fluidic channel.

A kit of the present invention may comprise a sample testing device according to the first aspect, instructions for use, a control sample, and optionally and one or more of buffers, detectable particles, application means (such as pipettes), instructions, charts, desiccants, control samples, dyes, batteries and/or signal processing/display means.

One or more features of the sample testing device and/or combined capture and signal measurement zone may be as described herein with respect to the first and/or second aspects of the invention.

A kit may additionally comprise, materials and apparatus mentioned herein such as buffers, detectable particles, application means (such as pipettes), instructions, charts, desiccants, control samples, dyes, batteries and/or signal processing/display means.

A kit may also comprise a control element as described herein for integration with the device. A kit may also comprise a reader for wirelessly powering the device. A kit may also comprise one or more containers of fluid (e.g. wash buffer or substrate solution).

Methodology of Heterogeneous Capillary Assay (e.g. ELISA)

In a second aspect of the invention, there is provided a method of performing a heterogeneous assay in a capillary lumen of a capillary passage. In its broadest form, the method comprises the steps of:

(a) providing a sample testing device comprising:
(I) a capillary passage having a lumen, and serving to fluidly connect, in series:
i. a fluid application region at an upstream end of the capillary passage;
ii. a reagent zone comprising a signal-linked binding member;
iii. a capture zone comprising means to capture the signal linked binding member (a “bound” fraction);
   (b) adding sample to the fluid (preferably sample) application region and causing it to flow downstream by capillary action through the reagent zone, thus creating a mixture of sample and reagent including signal linked binding member;
   (c) adding a wash buffer and causing it to flow downstream in the capillary passage following the sample, such that any sample or reagent which is not retained by the capture zone (the “free fraction”) passes downstream through the capture zone;
   (d) detecting the signal of the captured signal linked binding member in the capture zone as a measure of the amount of analyte present in the sample.

Sample may be prevented from reaching the reagent until buffer is added. This has the advantage of the reaction only beginning when the buffer is added, and so reduces the time critical steps for the end user. This may be achieved by suitable operation of the control means.

In step (b) when sample is added to the fluid application region, any first sealing means may be operated to seal the outlet of the capillary passage and any second sealing means are operated to not seal the outlet of the associated side passage. Sample may flow along the capillary passage by capillary action only as far as the intersection with the side passage, because the outlet of capillary passage is sealed. Sample is, however, able to flow into and along the side passage because the side passage outlet is not sealed. The capillary will fill until all sample has been drawn in. Any excess liquid above the test volume will begin to fill the side passage. Flow stops when all sample has been drawn in from the fluid application region into the capillary passage (the back pull in the capillary then equaling the forward pull).

Step (b) may further comprise reversing the conditions of sealing, such that the capillary passage outlet is not sealed and the side passage outlet is sealed. The sample in the capillary passage is then free to flow further along the capillary passage, for example by capillary action. No further flow will take place along the side passage, including back-flow towards the capillary passage.

During step (b), any fluid flow control means are operated to allow capillary flow along the capillary passage, from the fluid application region, downstream.

Step (c) comprises the step of release of the wash buffer. In an embodiment, completion of sample metering may prompt the user to release chase buffer, for example by use of a detection zone which is activated when sample flows past. In an embodiment, the sample does not reach the reagent until buffer is added (for example, by suitable operation of the control means). Where fluid dispensing means are provided, step (c) may comprise operating fluid dispensing means to release wash buffer into the capillary passage. Where a second inlet is provided, the wash buffer may be released into the second inlet. In an embodiment, step (c) may comprise depressing a button or rotating a cap which causes a reservoir of wash buffer to move relative to puncturing means (e.g. spikes) such that the reservoir is punctured. In step (c), buffer is released and flows into the capillary passage behind the sample. In this way, a sufficient volume of liquid is available for flow to be maintained to the distal end of the capillary without the need for a large sample volume.

As sample flows through the reagent zone by capillary action, the sample mixes with reagent of the reagent zone. The reagent includes a signal linked binding member, which in a 2-site or 1-site heterogeneous assay is a binding member which binds any analyte present. In a competitive assay, the signal linked binding member may be one which competes with analyte for binding to a binding member. In an embodiment, a further capture binding member may be provided in the reagent zone, which binds to analyte or signal linked binding member, and is retained by ligand-receptor immobilisation as it passes through the capture zone.

Capillary flow along the reaction zone allows sufficient time for any binding to occur.

For any heterogeneous assay, it is necessary to separate the bound and free fractions of the signal linked binding member so that the quantity of signal of one fraction (usually the bound fraction) can be measured and thus the concentration of analyte in the sample determined. In the present invention, separation of a free and bound fraction is performed by allowing flow of wash buffer to continue through the capture zone by capillary action, thus transporting any un-retained reagent and sample (including any signal linked binding member) through the capture zone, and downstream toward the outlet/fluid sump. Any fluid flow control means are operated during step (c) to allow continuous flow of liquid through the capture zone. Flow will stop when liquid reaches or fills the outlet and/or fluid sump. Thus, by defining the dimensions of the wash zone of the capillary the volume of wash fluid can be accurately and reproducibly defined without the need for pumps, valves, dispensers, operator intervention, etc.

Step (c) may further comprise the addition of substrate, where the signal is an enzyme or catalyst, and a measurable signal is generated upon reaction with a substrate (for example, in an ELISA). In an embodiment, a substrate solution may be added following release of wash buffer into the capillary. Where fluid dispensing means are provided for a substrate solution, step (c) may comprise operating the fluid dispensing means to cause substrate solution to be released into the capillary passage via a first or second or further (e.g. third) inlet, such that it flows along the capillary passage following wash buffer. Flow may be determined by a detector region in the capillary, providing an indication when flow of wash buffer has stopped, and substrate may be added. The user is prompted to release the substrate which flows into the capillary behind the wash buffer.

In an alternative embodiment, the wash buffer may comprise any substrate, such that release of a second liquid is not required, thereby simplifying the assay format.

In this embodiment, the invention provides the advantage of combining the processes of free/bound separation and addition of substrate into a single step, requiring only the release of buffer into the capillary passage by the user.

Fluid flow is detected by detection means at the end of the fluid sump or at the end of the capillary passage, prompting the initiation of a defined time period for any signal to develop and to measure signal of the bound fraction. Prior to cessation of fluid flow, any signal generated from reaction of signal linked binding member and substrate (e.g. during reaction in the reaction zone or after capture) will be washed away along with unbound enzyme reagent.

Once the detector has determined that substrate has reached the end of the capillary track, the signal measurement system is initiated, followed by data reduction and display of the calculated result.

The method of the invention comprises a washing step in which unwanted, unbound excess reagents are washed from the capture zone, downstream toward a fluid sump. In an embodiment, any enzyme substrate is continually washed through the capture zone, including any substrate that has changed colour. Only substrate which is retained in the capture zone due to cessation of flow by virtue of the fluid having reached the end of the capillary track will accumulate coloured product which is the signal for the assay. For accuracy, therefore, a signal measurement step is not performed until washing is complete. Alternatively, signal may be measured during all or part of the washing process, for example for control or calibration purposes.

In an embodiment, step (d) comprises allowing a time period to elapse between completion of fluid flow and measurement of signal. In an embodiment, step (d) comprises passing light through the capture zone, and detecting change in absorbance or reflectance by operating a photodetector.

The method of the invention may further comprise the step of converting the measurement of light absorbance or reflectance to a measurement of analyte concentration.

In embodiments where a further capture zone is provided, step (d) may be repeated for additional measurements of signal generated by the free fraction.

The method of the invention may comprise the steps of moving a control element between first, second, third and fourth position, as described above, to control fluid flow through the capillary passage.

In an embodiment, the method may comprise providing a combined capture and signal measurement zone as an insert; and integrating the insert with a capillary passage of the sample testing device. Preferably, the signal measurement is performed across the signal measurement zone, as described herein.

### Signal-Linked Binding Member

A signal linked binding member as defined herein comprises a member of a binding pair (e.g. antibody, analyte, analogue etc., as defined herein) conjugated to a signal. The signal may be a direct signal, which can be observed without the need for any additional reagent or reaction. Alternatively, the signal may be one which is generated, for example by action upon a substrate. Thus, a signal may be a coloured particle (for example, colloidal gold), a fluorescent molecule. Alternatively, it may be an enzyme or catalyst, which reacts with a substrate to generate a measurable output. The signal may be directly or indirectly linked to a binding member. Where the signal is generated, the term “signal” herein refers to the enzyme or catalyst label on a binding member, and also to the signal generated by reaction between the enzyme or catalyst and its substrate, which is then measured.

Any suitable signal may be used, many examples of which will be known and available to persons skilled in the art. Preferred signals are those that can be detected in the electromagnetic spectrum, such as chromophores and fluorophores, and enzyme/substrate systems such as Horseradish peroxidase/TMB. Others will be known to persons skilled in the art. In the latter case, a binding member may be bound to an enzyme, which catalyses the signal substrate to produce a colorimetric output. Preferred signals are those which employ an amplification system. Enzyme labels which can act on a substrate to produce chromophores are most preferred, e.g. Horseradish Peroxidase, alkaline phosphatase, beta galactosidase. Suitable substrates include TMB ABTS, OPD (for HRP), pNPP (for AP) and ONPG (for beta galactosidase).

In an indirect detection method, a binding member may be linked to a ligand-receptor pair, one of which is conjugated to an enzyme, as described above.

In a further embodiment, it is possible to use an unlabeled analyte binding member, with an enzyme-coupled or biotinylated secondary antibody which binds the analyte binding member. Such an embodiment enables greater signal amplification than direct labelling of the analyte binding member. If the secondary antibody is biotinylated, then a tertiary step is required for detection. In this case treatment with the streptavidin-enzyme conjugate, followed by an appropriate substrate.

The features and embodiments of each aspect applies to the other aspects of the invention, mutatis mutandis.

### EXAMPLES

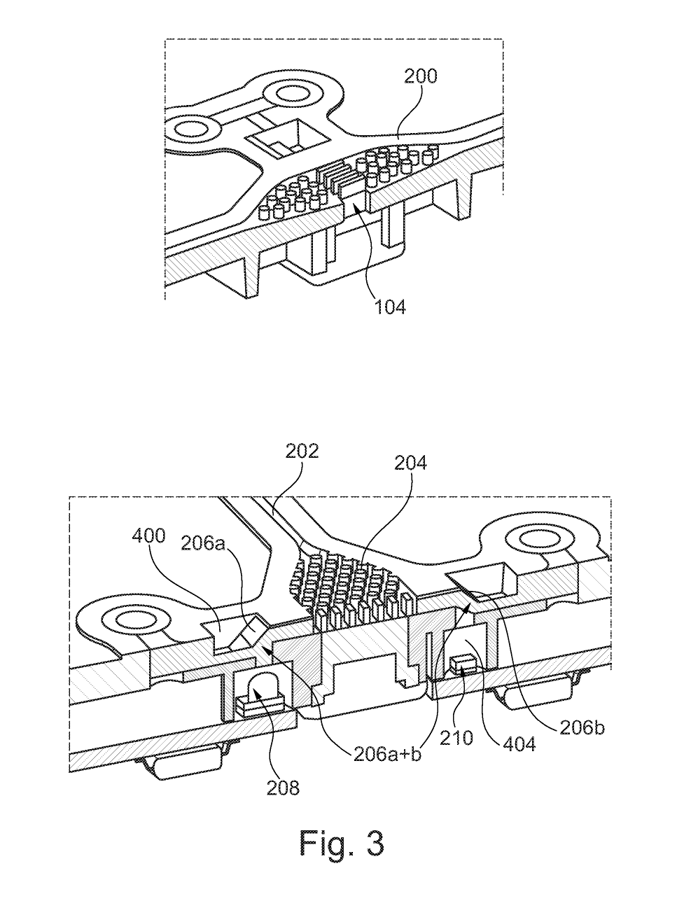

In one example, in accordance with an embodiment of the present invention, a sample testing device (also referred to as a capillary pathway device or a chip device) 300 including a combined capture and signal measurement zone (SMZ) 200 is shown in FIG. 3. The combined capture and signal measurement zone 200 includes a series of transparent parallel “fins” 104 aligned parallel to the direction of flow in a broadened area of a capillary passage (also referred to as a track or pathway) 202.

The fins 104 are elongate and define fluidic channels 103 therebetween for receiving fluid from the capillary passage 202. The lengths of the fins 104 are substantially parallel to one another and the fins 104 are aligned with one another along a line that is substantially perpendicular to the lengths of the fins 104.

The fins 104 may be formed integrally with one or more other components of the sample testing device, or may comprise a separate insert 100 such as that shown in FIGS. 3 and 4. FIG. 4 shows a detailed view of the insert 100 according to an embodiment of the present invention. The plurality of elongate fins 104 are upstanding from a body of the insert 100. Additionally, the insert 100 includes a flanged section 106 that facilitates the locating of the insert 100 in a capillary pathway device. In alternative embodiments, the insert 100 may include other mechanisms and/or features (or none at all) for facilitating the locating of the insert 100 in a sample testing device.

The combined capture and signal measurement zone 200 includes an optical pathway 400 for measurement of the fluid therein. The fins 104 are arranged substantially perpendicularly relative to the optical pathway 400. Additionally, the elongate fins 104 are configured to permit optical transmission therethrough along the optical pathway 400 so that optical radiation can pass through the fins 104 and fluid in the fluidic channels between the fins 104 so that attenuation may be measured. In particularly preferable embodiments, the fins 104 are entirely optically transparent so as to minimise any attenuation of the optical radiation caused by the fins 104.

The fluidic channels 103 defined by the fins 104 serve (via an immobilised capture reagent) to bind an immune complex formed in the reaction zone and retain it during the wash step. When the bound complex is incubated with substrate, signal (e.g. colour) is generated in the spaces (fluidic channels 103) between the fins 104. This signal can be measured by directing light across the SMZ (along the optical path 400) and through the fins 104, quantifying the signal in the spaces between the fins 104. The use of transparent fins 104 parallel to the sides of the SMZ (and perpendicular to the direction of the optical pathway) minimises interference in the measurement process.

The above-described arrangement offers significant benefits over existing designs, including:
provision of a large surface area for capture of bound signal fraction in the fluidic channels defined by the fins 104
minimal resistance to flow for efficient washing
a long optical pathway 400 to increase sensitivity.
a short, mean-free path for substrate-enzyme reaction.

The broadened area of the capillary 202 has essentially parallel sides with a width of 1-20 mm, ideally 3-10 mm. To ensure continuity of fluid flow there is a tapered region 203 leading into and out of the read/capture zone 200 linking it to the main capillary passage 202. Features (e.g. micropillars 204 with a height of 1.02 mm and a diameter of 0.5 mm) may be incorporated into the tapering zones to assist fluid flow and minimise formation of bubbles, etc. which could affect the optical pathway 400 or reduce wash efficiency.

The embodiment where the fins 104 are provided on a removable insert, such as the insert 100 shown in FIG. 4, allows for separate production of the capillary device 300 and any treatments to be performed independently of the SMZ 200 or on the insert 100, greatly simplifying manufacture.

Any mechanism can be employed for directing light across the SMZ 200 along the optical pathway 400. In one preferable embodiment (as shown in FIG. 3) prism-shaped “windows” 206 within the device 300 are arranged to redirect optical radiation (e.g. light) through 90°. This allows an optical source 208 and a detector 210 to be surface-mounted on the device 300 yet still provide optical radiation along the optical pathway 400 through the SMZ 200. In the embodiment shown in FIG. 3, the windows comprise a first prism 206a positioned at a first end of the optical pathway 400 and a second prism 206b positioned at a second end of the optical pathway 400. The first prism 206a is configured to redirect optical radiation from the optical source 208 along an emission pathway 402 so that it travels along the optical pathway 400 through the SMZ 200. Similarly, second prism 206b is configured to redirect optical radiation travelling along the optical pathway 400 (subsequent to travelling through the SMZ 200) and redirect it along a detection pathway 404 towards the detector 210. Whilst the first and second prisms 206a, 206b shown in FIG. 3 redirect optical radiation by 90°, the prisms 206a, 206b may redirect optical radiation by other non-zero angles within the scope of the present invention. In the embodiment shown, the prisms 206a, 206b redirect light by total internal reflection (TIR) at the prism-air boundary which is orientated at 45° relative to the incoming pathway (e.g. emission pathway 402 for the first prism 206a, and optical pathway 400 for the second prism 206b) in order to redirect the light through 90°.

Whilst preferable embodiments will include both the fins 104 described above and the prisms 206a, 206b described above, both arrangements provide independent benefits. Certain aspects of the present invention may therefore comprise either arrangement without necessarily incorporating the other, as defined in the appended claims.

In preferable embodiments, any one or more of the optical components (fins 104, prism-shaped windows 402, 404 etc.) are moulded from a transparent plastics materials, such as polycarbonate, whilst the device 300 is moulded from an opaque plastics material (e.g. polycarbonate containing a black dye) to prevent stray light interfering with the measurement process.

The combined capture and signal measurement zone 200 provides an optically clear test chamber. In order to observe the highest change in optical properties of a sample, the optical path length should be as long as possible in the sample. However, this must be balanced with the need to deposit a sufficient quantity of reagent in the observation area. That requires for a larger surface area than a typical empty chamber could provide.

The insert 100 with fins 104 described above, provides an effective solution. The fins 104 reduce the optical path length over which light passes through the sample liquid, but significantly increases the surface area for reagents. These reagents, by a process of chemical bindings (not within scope of this application) provide catalyser sites for the colour change reaction. The colour change occurs in the solution around the reagent coated surfaces. Further, by interposing the fins 104 at intervals throughout the liquid, the mean free path of the reaction between substrate in the liquid phase and enzyme (immobilised) is reduced, thus increasing the rate of reaction.

In one embodiment, the fins 104 are moulded in plastic (e.g. polycarbonate) and may consequently have tapered surfaces, being wider at their respective bases 104a compared with their respective tips 104b.

The fins 104 present provide a greater surface area for reagents, which results in faster reactions and larger colour change signals to measure. The total number, shape and dimensions of the fins 104 should therefore be chosen such that a sufficient colour signal may be obtained whilst increasing the surface area for reagents by a desired amount.

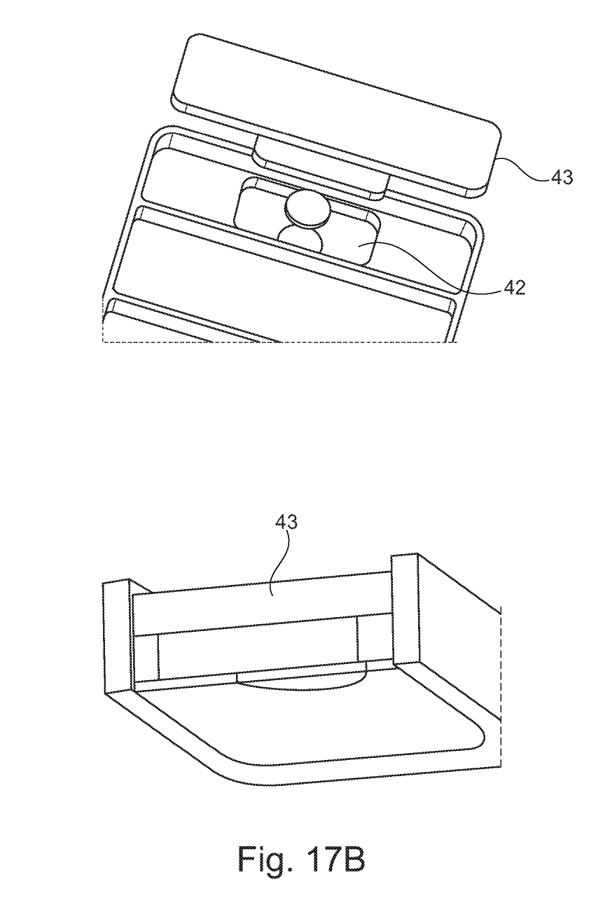

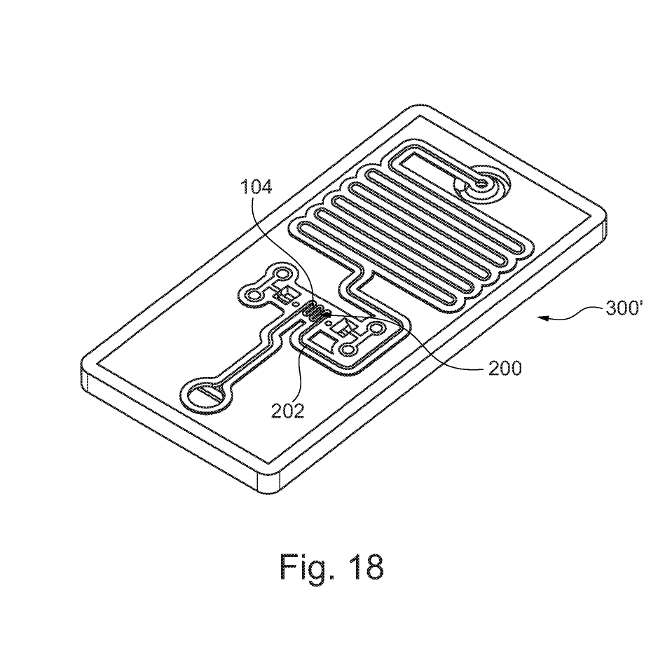

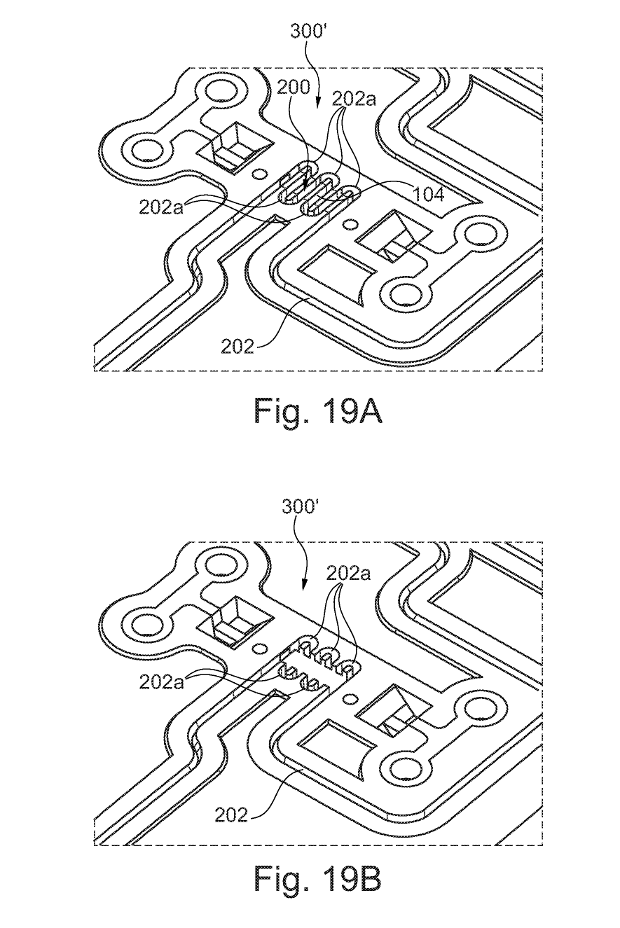

FIG. 18 shows an alternative device 300 in accordance with an embodiment of the present invention. The device 300″ is identical to the device shown in FIG. 3 with the exception of the fluidic connection between the capillary passage 202 and the fins 104 in the SMZ 200. As described above, in the embodiment of FIG. 3, the capillary pathway 202 broadens so that fluid travelling along the capillary passage passes substantially simultaneously through each of the fluidic channels 103 defined by the fins 104. In contrast, in the embodiment shown in FIG. 18, the capillary passage 202 includes a series of looped portions 202a that direct fluid travelling along the capillary passage 202 sequentially through adjacent fluidic channels 103 defined by the fins 104. FIG. 19A shows this fluidic arrangement in more detail where it can be seen that the capillary passage 202 is directed to a single fluidic pathway alongside one of the fins 104. The looped portions 202a of the capillary passage 202 create a fluid path between adjacent fluidic channels 103, and, downstream, the capillary passage provides a fluid path away from the SMZ 200.

As with the embodiment described above in relation to FIG. 3, the fins 104 of the embodiment of FIGS. 18 (and 19A) may be formed as part of an insert (e.g. such as that described above in relation to FIG. 4), or they may be formed integrally with one or more other components of the capillary pathway device 300′.

FIG. 19B shows the capillary pathway 300′ in accordance with an embodiment of the invention in which the fins 104 form part of an insert, where the insert is removed and the fins 104 are not present. As FIG. 19B shows, in such an embodiment, without the fins 104 present, the capillary passage 202 does not form a continuous fluid path and instead includes a series of disjointed looped portions 202a.

The embodiments described above in relation to FIGS. 18, 19A and 19B offer certain advantages over alternative arrangements. In particular, the nature of the capillary pathway 202 provides a longer path length for the fluid and so increases contact time with the fins 104, and may improve washing efficiency by eliminating possible “dead-spaces”.

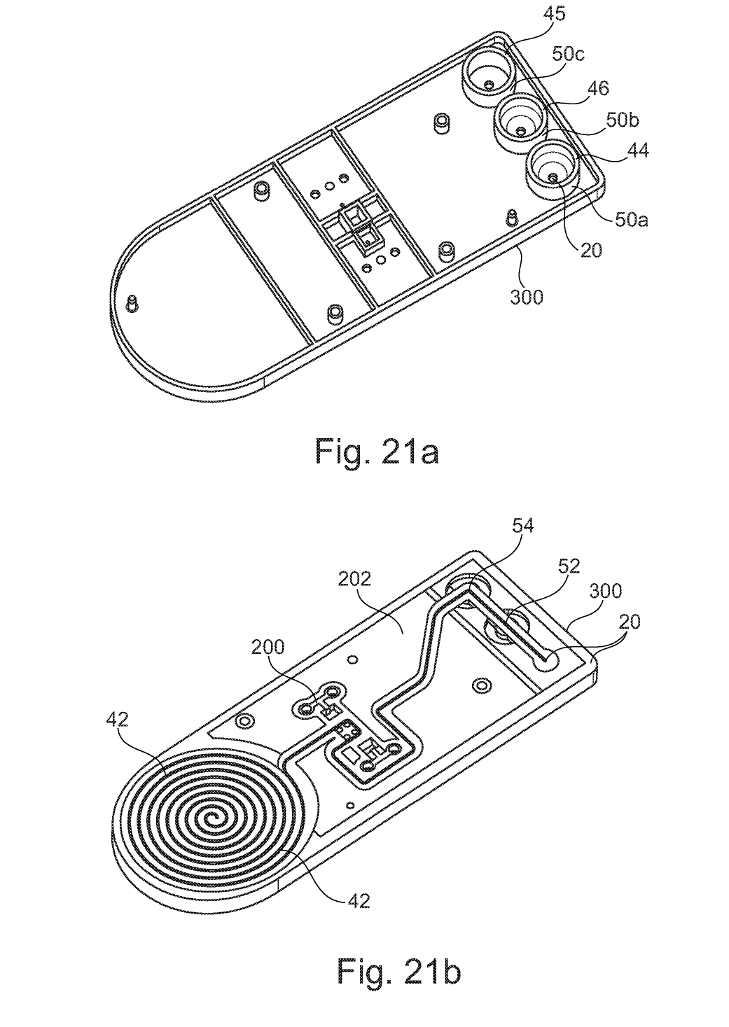

FIG. 21a shows a surface of a device 300 of the invention. Wells 44, 46 and 48 are shown comprising upstanding collars 50a, 50b, and 50c, and having an inlet 20, 52 and 54 located centrally within a collar. A first inlet 20 is provided for sample application to capillary passage 202. In FIG. 21b, the inlets 20, 52 and 54 are seen on the opposite surface of the device 300. A single capillary passage 202 extends from first inlet 20, to inlets 52 and 54 which are connected in series by capillary passage 202. The inlets and capillary passage run parallel to a shorter outer edge of device 300. The capillary passage 202 runs toward the centre of the device to SMZ 200, and then toward fluid sump 42′. Fluid sump 42 comprises two capillary passages which branch from passage 202 and which run in parallel in a spiral configuration.

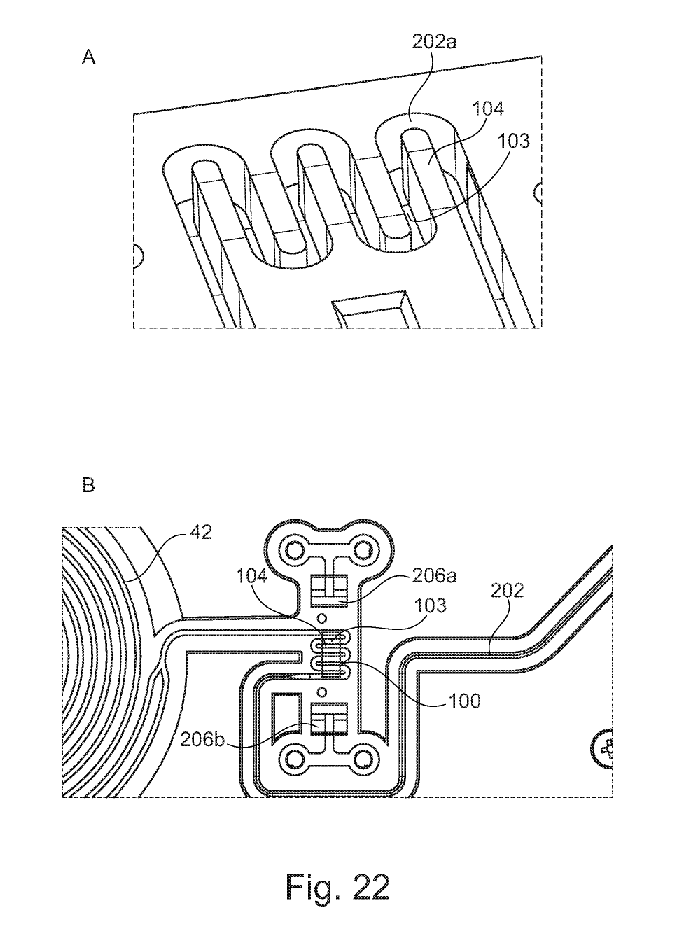

FIG. 22 shows the SMZ 200 in detail, where disjointed looped portions 202a and fins 104 together define a serpentine path for the capillary passage 202. Fluidic channels 103 extend between fins 104. The rectangular position 100 outlines the insert comprising fins 104.

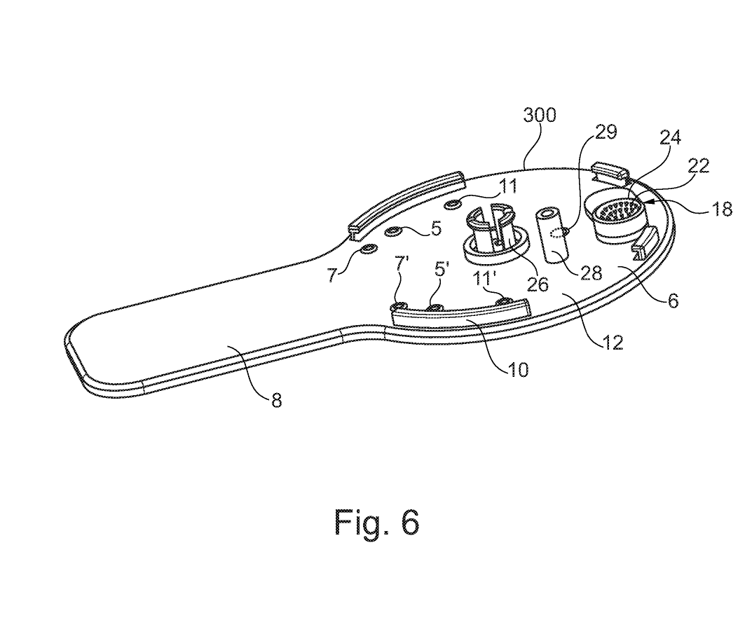

In another embodiment, a device according to the invention is shown in FIG. 6, and comprises a rigid, planar plate of injection moulded polycarbonate, having a circular head portion 6 and an elongate tail portion 8 extending therefrom. The device is formed with an upstanding outer collar 10 on the upper surface 12 thereof.

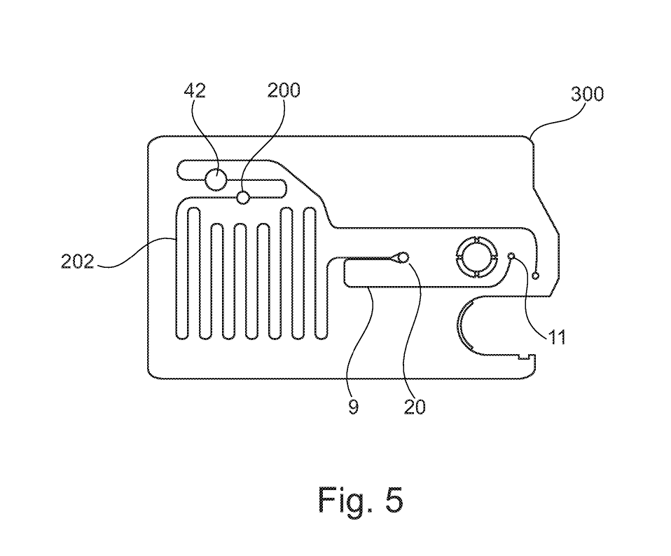

As seen best in FIG. 5, the outer collar 10 is located in the circular portion of the sample metering element 2 and includes part-circular portions constituting part of a circle having a radius of about 32 mm. The outer collar 10 works in conjunction with the inner collar 26 and is provided to retain in place a control element 4 on the upper surface 12.

The upper surface 12 includes a circular, funnel-like, recessed portion 18, leading to an inlet. The funnel-like recessed portion 18 comprises micropillars 22 extending downward from the inside surface 24 of the recessed portion 18. The micropillars 22 help to draw the sample into the sample application region and also aid the flow of the sample toward the capillary passages 202. The upper surface 12 further comprises an upstanding inner collar 26 formed of four part-circular sections, which form both a retaining feature and a pivot point about which the control element 4 turns. The pivot point is located centrally within the circular portion 6 of the device 2. The upper surface 12 of the device 2 further comprises an upstanding post 28 which serves to hold buffer release capsule 30 in place during puncturing. Through hole 29 is provided in upper surface 12 for fluid to flow from buffer release capsule 30 into a second inlet on the lower surface of the device 2.

A single capillary passage 202 extends from a first inlet 20. Each track includes an overflow passage 9, extending as a side branch perpendicular from the associated main track 202 and turning through 90° to extend firstly back towards the first and second inlets 20, 32, and then turning through 45° to extend in a direction toward the outer edge of the device 2. An overflow passage 9, terminates in an outlet 11, which is open on the upper surface 12 of sample metering element 2. A side (overflow) passage 9 may be wider than a main passage.

A main passage 202 is V-shaped in section and have the cross-sectional profile of an equilateral triangle with sides 0.435 mm long. The depth of these passages is 0.377 mm. The overall length of each main channel is approximately 200 mm. An overflow passage 9 is trapezoidal in cross section, having a flat base 0.3 mm in length with outwardly inclined side walls defining an angle of 60° therebetween. The depth of these passages is 0.38 mm.

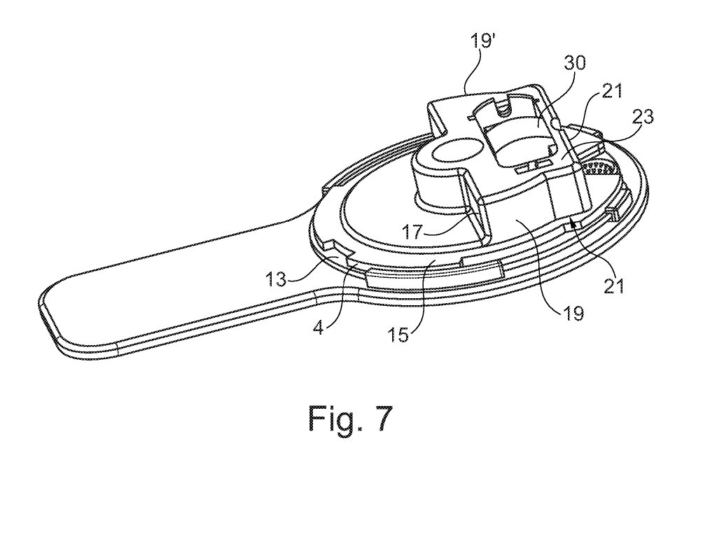

As shown in FIG. 7, a control element 4 can be fitted to the device 2. As shown in FIG. 7, the control element 4 comprises a generally circular planar, rigid first portion 13 of injection-moulded acrylonitrile butadiene styrene (ABS) with a diameter of about 63 m and a height of about 1.2 mm. The height refers to the thin flange of circular portion 13. Overall the height of the control element from the base to the top is approximately 13.5 mm. The circular first portion 13 comprises sealing means (not shown) on the underside, which is in contact with the upper surface 12 of the device 2. The generally circular first portion 13 also comprises cut out sections to reveal or shield (or seal) the funnel-like sample entry port 18, such that in a third or fourth and fifth positions as defined above when sample has entered the channels, access to the funnel-like sample entry port is closed to the user. The opening or closing of the sample entry port 18 is actioned by rotating the control element about the pivot 26 provided on the sample metering element 2.

The circular planar first portion 13 is stepped to second portion 15 which comprises a semi-circular portion of smaller diameter than the first portion 13. A first upstanding wall 17 extends along the straight edge of the semi-circular portion, and defines an inner semi-circle centrally on the straight edge, thus defining a planar “C” shape. The inner semi-circular wall 17 defines a recess about the pivot point which upstands from the upper surface 12 of the element 2. Side walls 19, 19′ extend to follow the circumferential edge from the ends of first wall 17, and an end wall 21 is provided to define with the first wall 17 and side walls 19, 19′, a generally rectangular housing 21 which houses buffer release means. A lid 23 is provided to close the buffer release means housing.

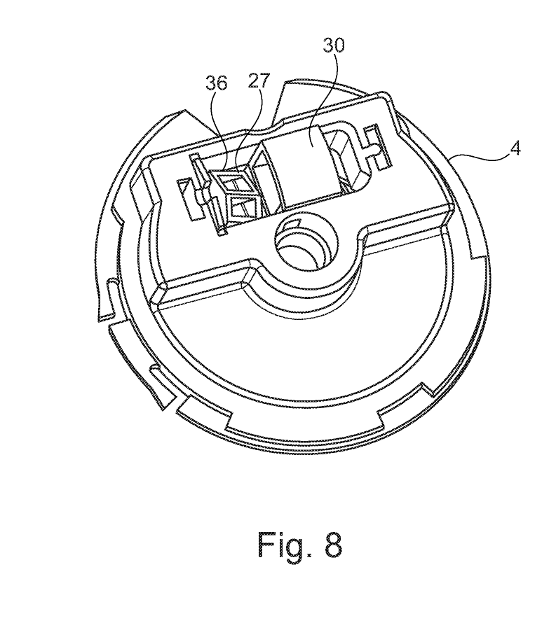

The substantially rectangular housing 21 comprises an arcuate cover 25 (FIG. 9). Within the housing is provided a buffer release capsule 30 which is held in placed by post 28. As shown in FIG. 8, rupturing (or piercing) means 36 are provided on a planar element 31 which sits against an inner surface 33 of side wall 19′. A cam is provided (not shown) such that rotation of the control element causes the puncturing means 36 on planar element 31 to move toward capsule 36 and drive into it. The rupturing means 36 comprise a series of fins 27 which extend outwardly, and which are joined together at a centrally defined point which in an active position can intersect the fluid filed polypropylene capsule 30 which is dimensioned to fit snugly within the housing 21. Thus, the rupturing means 36 are movable between a first, ready position, and a second activated position by application of a suitable rotational force to the rupturing element. The force causes the capsule 30 to be punctured with consequential release of the fluid contents.

A cylindrical soft rubber seal 40 of thermoplastic elastomer (TPE) with a Shore hardness of 40A is fitted into the grooves standing slightly proud of the lower surface of the control element 4, forming sealing members that cooperate with the capillary passage outlets 5, 5, 7′, 7′.

A sheet of flexible foil 106 in the form of a clear polycarbonate sheet 0.06 mm thick is secured by laser welding to the lower surface 16 of the device 2 to cover the passages 202, 9 and convert them into enclosed capillary passages, also referred to herein as capillary pathways.

Hydrocarbonates such as ABS or polycarbonates are hydrophobic which means that aqueous fluids will not flow well within the passages. To address this, the capillary passage internal surfaces are treated to provide a thin coating of Tween 20 surfactant (Tween is a Trade Mark) to impart hydrophilic properties to the capillary surface. This can be done by any suitable means, for example using a vacuum process to draw a solution of Tween 20 in deionised water (comprising 0.5% by volume Tween 20) through the capillary passages, by applying suction at an open end of the passages or by dip tweening.

This treatment also performs a quality control function in that it will reveal if any of the capillary passages are blocked, e.g. as a result of imperfect moulding, imperfect sealing of the foil, or the presence of debris or foreign matter in the passages, enabling defective elements to be discarded at this stage.

Prior to use, control element 4 (see FIG. 7) is located on the outer collar 10 of device 2, with the control element 4 in a first position, where the device is in an inactive state. In the first position, the control element 4 is positioned such that the sample entry well 18 is shielded/sealed by the planar circular portion 13 of the control element 4, so cannot be used and is also protected from ingress of foreign material. None of the passage outlets 5, 5′, 7, 7′ are sealed.

The device in this condition may be packaged for distribution and sale, e.g. being sealed in a foil pouch which is impermeable to air and moisture.

When the device is required for use, the control element 4 is rotated to a second position. In this position, the planar circular portion 13 is positioned such that the sample entry well 18 is exposed, and sample can enter the sample entry hole 20 of the element. In addition, the main passage outlets 5, 5′, 7, 7′ are sealed by portions of the seal 40, while the overflow passage outlet 11 are not sealed.

A quantity of fluid sample e.g. a blood sample to be tested (possibly containing an analyte of interest) is added to the device via sample entry hole 20. It is important that more sample is added than is required for the test, with a sample of about 15 microlitres being appropriate in the present case. The sample fluid flows along the initial portions of a passage 202 and then into the overflow passages 9. The sample cannot flow further along the main passage 202 because the main channel outlets 5, 5′, 7, 7′ are sealed by the seal 40 of the control element 4. In this way, a defined quantity of sample is present in each of the main passages (referred to as the test volume), with excess being passing into the overflow passages. In the present embodiment, the test volume in each main passage is about 5 microlitres.

The control element 4 is then rotated through a third position (where the sample well 18 of the device 2 is shielded (sealed) by the planar circular portion 13 of the control element 4, the overflow channel outlet 11 and the main channel outlets 5, 5′, 7, 7′ are now sealed by seal 40, respectively to a fifth position where the sample well 18 remains sealed, the overflow channel outlets 11 remain sealed by seal 40, whilst the main passage outlets 5, 5′, 7, 7′ are not sealed.

Fluid in the capsule is then introduced to the capillary passages 3, 3′. Typically the fluid is a chase buffer, e.g. PBS, which enables the reaction to be carried out with a smaller volume of sample than is required to flow around the entire capillary system to determine a test result. This is achieved by operation of the rupturing means 36.

Rotation of control element and 4 causes movement of rupturing means 36 into the activated position, resulting in piercing of the capsule by the point 36, and release of fluid from the capsule to flow into the second inlet 32. In the preferred embodiment shown, this is achieved by rotation of the cap 4 between positions 2 and 4 which causes the rupturing means 36 to move relative to the capsule 30 which is retained by post 28.

The capsule fluid e.g. wash buffer, pushes the test sample further along the main passages, 3, 3′.

Sample (followed by chase buffer) will flow along the main passages, by capillary flow. Because the overflow passage outlets 11, 11′,′ are now sealed, no further flow will take place along the overflow passages 9, including no back-flow towards the main passages. Instead, fluid flow will be along the main passages, 202, towards the unsealed main passage outlets 5, 5′, 7, 7′. The sample will thus flow past the reagent zone in the passage 202.

Control element 4 is operated to allow continuous flow of liquid through the capture zone. Flow will stop when liquid reaches or fills the outlet and/or fluid sump 42. Thus, by defining the dimensions of the wash zone 212 of the capillary the volume of wash fluid can be accurately and reproducibly defined without the need for pumps, valves, dispensers, operator intervention, etc.

In an embodiment, a substrate solution may be added following release of wash buffer into the capillary. Where fluid dispensing means (30, 36) are provided for a substrate solution, this step may comprise operating the fluid dispensing means to cause substrate solution to be released into the capillary passage 202, such that it flows along the capillary passage following wash buffer. Flow may be determined by a detector region in the capillary, providing an indication when flow of wash buffer has stopped, and substrate may be added. The user is prompted to release the substrate which flows into the capillary behind the wash buffer.

Fluid flow is detected by detection means at the end of the fluid sump 42 or at the end of the capillary passage, prompting the initiation of a defined time period for any signal to develop and to measure signal of the bound fraction. Prior to cessation of fluid flow, any signal generated from reaction of signal linked binding member and substrate (e.g. during reaction in the reaction zone or after capture) will be washed away along with unbound enzyme reagent. An absorbent pad 43 may be provided within the fluid sump 42.

Once the detector has determined that substrate has reached the end of the capillary track, the signal measurement system is initiated, followed by data reduction and display of the calculated result. An LED 208 is used to pass light along light path 400, via prisms 206a, b, which direct light across the fins 104 and toward the detector 210.

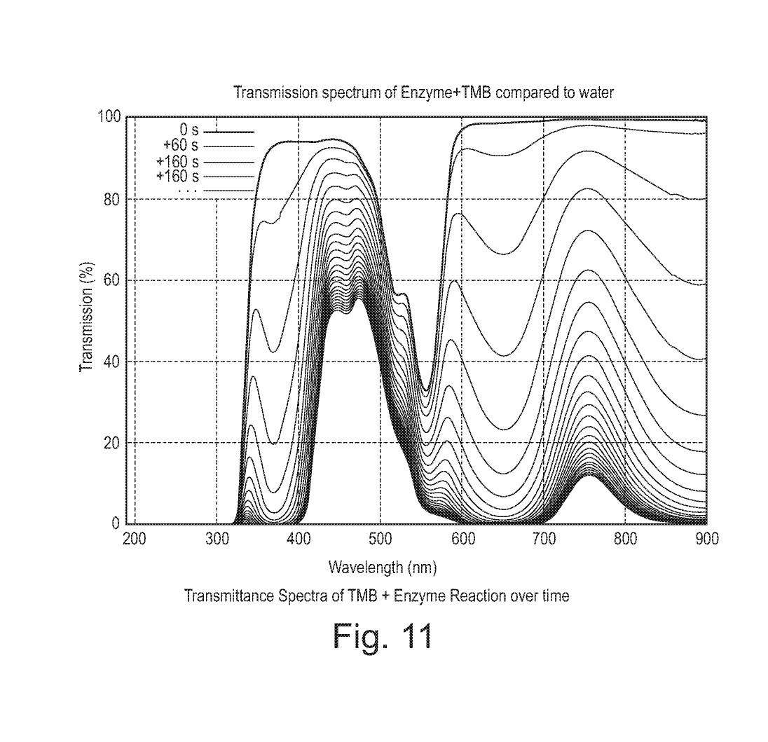

FIG. 11 shows spectra obtained for the reaction of TMB (substrate)+Enzyme (catalyst). TMB changes from pink to blue in the presence of the enzyme. This principal can be extended to cover many other biochemical substrates and “signals”.

Note that due to the spectro-photometer equipment used, there is a sweep time of 60 sec. This means that the data is skewed linearly in time by 60 secs from the left to the right of the graph.

It is useful to identify multiple wavelengths of significant “activity” in the preceding graphs and to observe the change in transmission or absorption at these wavelengths as time progresses. 1 to 3 wavelengths can be identified as being practical and cost effective. The use of multiple wavelengths costs more but offers significant advantages in the calibration of readings and potentially better reliability under fault conditions. Ideally a wavelength is identified that is unaffected by the colour change but as this cannot be done in all cases (for example the case of TMB as a biochemical substrate) wavelengths are considered which have minimum change over time, As well as at least one wavelength for which there is a maximum degree of change. In the case of TMB, 370 nm, 460 nm, 650 nm and 900 nm are of interest. However since 470 nm (blue), 625 nm (red) and possibly 530 nm (green) are commercially available co-mounted as surface mount RGB LED components; these have been used for development.

In this particular configuration of ELISA (i.e. the set of biochemical reagents and biochemical “signals”) the colour change is observed in a solution and so is mainly optically transmissive and absorptive rather than reflective. So using TMB+Enzyme to generate a biochemical signal we observe changes in optical transmission.

The following example contains data supporting the conversion of a conventional enzyme-linked immunosorbent assay (ELISA) to a linear microfluidic approach, suitable for a point-of-care format

### 1. Simultaneous Fluid Phase Reaction Between Signal and Capture Antibodies and Analyte (Signal Detection at 370 nm).

One of the key requirements facilitating the performance of ELISA type assays in a one-way linear microfluidic format is the ability of the assay analyte and reagents (capture and signal antibodies) to react simultaneously in fluid phase, forming antigen-antibody complexes, and the subsequent immobilisation of these complexes onto the solid phase of a coated detection zone. This approach differs from the standard ELISA approach, where each of the individual binding events between antigen and antibodies are performed sequentially at a solid phase (microtitre plate surface), where the capture antibody is bound.

An additional reduction in assay complexity, which is desirable for a point-of-care assay format, was to negate the requirement for an acidic “stop” solution at the end of the signal development phase. In a conventional ELISA this halts the signal development and converts the TMB signal from blue to yellow, which is measured spectrophotometrically at 450 nm. The examples below demonstrate the feasibility of using the blue colour as a more direct assay endpoint, at a fixed timepoint, by measuring light absorption at a wavelength of 370 nm.

The feasibility of the simultaneous fluid phase reaction approach and elimination of the Stop reagent was demonstrated using alpha-GST ELISA kit reagents (Argutus Medical) with biotinylated capture antibody (Fleet Bioprocessing) and is described below.

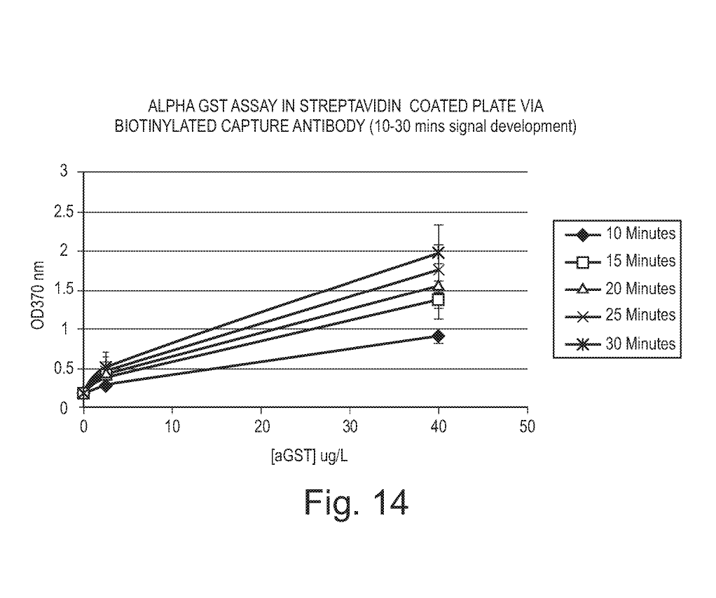

Reactions were performed using 50 ul each of a 1/10 dilution of stock HRP-labelled alpha-GST signal antibody and 23 ug/ml biotinylated anti alpha-GST capture antibody in kit conjugate diluent and 0, 2.5 and 40 ng/ml alpha-GST calibrator in sample diluent. Reactions were allowed to proceed at room temperature for 15 minutes then transferred to a streptavidin-coated microtitre plate and further incubation for 15 minutes. Wells were aspirated and 250 ul kit wash solution added. This step was repeated three times, followed by addition of 100 ul of TMB solution per well. Signals were measured at 370 nm using a spectrophotometer over a period of 30 minutes development time (FIG. 14).

### 2. Simultaneous Fluid Phase Immuno-Reaction Using Desiccated/Reconstituted Capture and Signal Antibodies (Signal Detection at 370 nm).

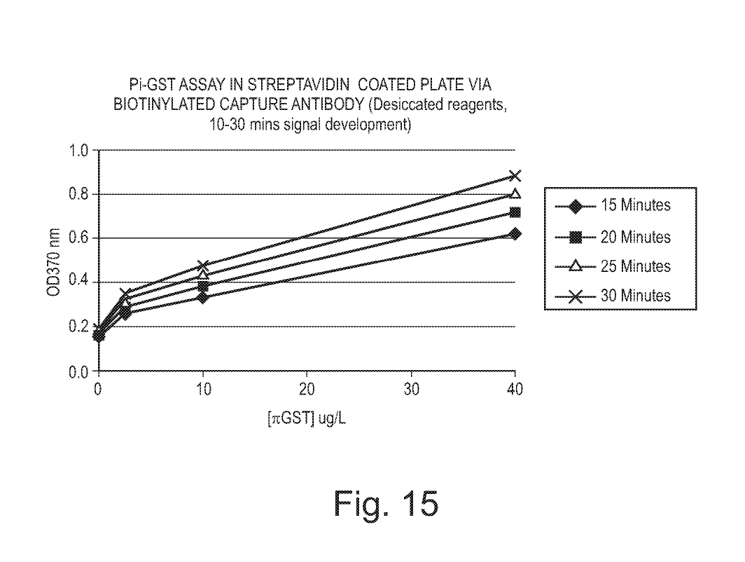

The feasibility of the simultaneous fluid phase immuno-reaction using desiccated/reconstituted capture and signal antibodies was demonstrated using pi-GST ELISA kit reagents (Argutus Medical) with biotinylated capture antibody (Fleet Bioprocessing). Capillary passages were prepared containing 1 ul each of anti-pi GST HRP-conjugate (stock) and biotinylated anti-pi GST capture antibody (0.3 mg/ml). Passage were dried thoroughly in a desiccated chamber at room temperature. Reagent reconstitution and assay reactions were initiated by the addition of 200 ul of kit sample diluent containing 0-40 ng/ml pi-GST and allowed to proceed at room temperature for 10 minutes. Reaction mixtures were then transferred to a streptavidin-coated microtitre-plate (Perbio Science UK) and allowed to incubate for a further 20 minutes at room temperature. Wells were aspirated and washed three times with 200 ul 10 mM sodium phosphate buffer solution pH7.4 containing 0.1% tween 20, followed by addition of 100 ul of TMB solution. Signals were measured at 370 nm using a spectrophotometer over a period of 30 minutes development time (FIG. 15).

### 3. Development of Combined Capture/Read Zone.

The signal measurement zone of the optical module features a measurement zone with maximized surface area, where analyte-containing immuno-complexes are immobilized and a coloured signal developed and measured, whilst minimising volume. In addition to maximizing the available area of optical read surfaces, the size and shape of the signal measurement zone must be of appropriate dimensions to support fluid flow by capillary forces alone.

As a design precursor experiment to enable suitable sized and shaped internal capillary features to be investigated, a set of prototype moulded polycarbonate microfluidic devices were produced and tested. The devices comprised a planar strip of injection-moulded polycarbonate measuring approximately 125×24×2 mm containing recessed circular areas measuring approximately 3 mm in diameter and 0.5 mm deep, joined by two v-shaped grooves of the same depth, so that when overlaid with a self-adhesive foil a continuous capillary passage was created. It was possible to introduce and remove fluids via either v-groove using a micropipette. Upstanding moulded cylindrical features, measuring approximately 0.5 mm high and of varied diameter and spatial arrangement, were positioned within the flat circular regions in order to increase the surface area and encourage capillary flow. Circular regions of the moulded devices were coated with avidin and their performance as capture/signal measurement zone assessed.

a) Coating Test Chips with Avidin.

Test devices were covered with self-adhesive tape above the recessed circular areas and for approximately 10 mm over the v-grooves on either side. The resulting capillaries were filled by pipette with 11 ul of 100 ug/ml avidin solution in 10 mM tris base and incubated at room temperature in a humidified container for three hours. After removal of the tape, the devices were washed three times in 10 mM sodium phosphate buffer pH7.4 containing 0.1% tween 20, followed by a final wash in 10 mM sodium phosphate buffer pH7.4 containing 0.25% tween 20 and 0.5% trehalose, then vacuum dried for 1 hour and stored in desiccation until required.

### b) Development of Assay Signals on Candidate Detection Zones.

Alpha-GST ELISA kit reagents (Argutus Medical) were used for the following experiment in conjunction with a biotinylated capture antibody (Fleet Bioprocessing) as described below.

Reactions were performed using 10 ul each of a 1/100 dilution of stock HRP-labelled alpha-GST signal antibody in phosphate-buffered saline pH7.4, 2.3 ug/ml biotinylated anti alpha-GST capture antibody in 10 mM sodium phosphate buffer pH7.4 and 0, 2.5 and 40 ng/ml alpha-GST calibrator in stabilised/unstabilised urine. Reactions were allowed to proceed at room temperature for 30 minutes, during which time the avidin-coated devices were prepared by covering the recessed circular areas with self-adhesive tape extending for approximately 10 mm over the v-grooves on either side. The resulting capillaries were filled by pipette with 10 ul of reaction mix and incubated for a further 10 minutes. After removal of the tape, the devices were washed three times in 10 mM sodium phosphate buffer pH7.4 containing 0.1% tween 20 and blotted dry. Self-adhesive tape was reapplied, 10 ul TMB solution introduced to each capillary and signals allowed to develop for 10 minutes in the dark. Signals intensities were judged visually by blue colour intensity on a scale of “+” (very light blue) to “++++” (dark blue) (Table 1).

TABLE 1

| Alpha-GST concentration | Stabilised urine | Unstabilised urine |
|---|---|---|
| (ng/ml) | (signal intensity) | (signal intensity) |
| 0 | + | + |
| 2.5 | + | ++ |
| 10 | ++ | +++ |
| 40 | +++ | ++++ |

### 4. Pi-GST Assays Using a Prototype Capillary Device (Pipetting Method).

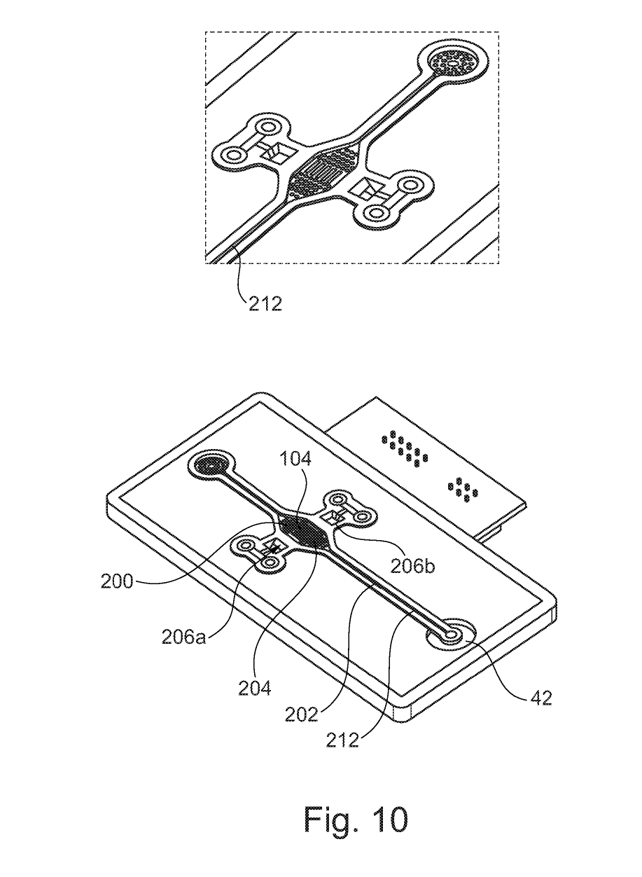

Pi-GST assays were performed in prototype devices (FIG. 10) using pi-GST ELISA kit reagents (Argutus Medical) in conjunction with a biotinylated capture antibody (Fleet Bioprocessing) as described below.

Prototype devices were prepared for assay use as follows.

Fin components (FIG. 4) were prepared by applying a coating of streptavidin as follows. Fins were incubated in 10 mM sodium phosphate buffer pH7.4 containing 100 ug/ml streptavidin for 3 hours at room temperature with constant mixing by inversion. Fins were then washed three times in 10 mM sodium phosphate buffer pH7.4 containing 0.1% tween 20 and 1% BSA. A final wash was performed in 10 mM sodium phosphate buffer pH7.4 containing 0.25% tween 20, 0.5% trehalose and 1% BSA. Streptavidin-coated fins were dried under vacuum for approximately 60 minutes then stored in desiccation at 2-8° C. until required.

Capillary devices (FIG. 10) were prepared for assay use by subjecting them to plasma-treatment to render the surfaces hydrophilic (Dyne Technology Limited). Reagents were applied to the devices in a 2-stage process; firstly 5 ul of 0.5% BSA/0.5% tween 20 was pipetted into the capillary v-groove (202) upstream of the fins (104) and desiccated at room temperature overnight. Secondly, equal volumes of 30.5 ug/ml biotinylated anti pi-GST capture antibody in 10 mM sodium phosphate buffer pH7.4 containing 1% sucrose and 1/100 dilution of stock HRP-labelled pi-GST signal antibody in 10 mM sodium phosphate buffer pH7.4 containing 1% sucrose were mixed and 8 ul applied to the device in the same position as the first stage reagents. Second stage reagents were dried under vacuum for 30-60 minutes, then stored in desiccation at room temperature until needed.

Devices were assembled by sealing the moulded capillary passages using self-adhesive tape and inserting a streptavidin-coated fin component into the central slot of the capillary device.

Assembled devices were slotted into a purpose-built electronic spectrophotometric rig, containing an LED light source and photodiode detector, with PC-based user-interface software. Transmission at 632 nm was monitored across the optical capture zone (SMZ) and the data recorded.

Test solutions were prepared by dilution of pi-GST kit calibrators in kit sample diluent to concentrations between 0 ng/ml and 40 ng/ml.

Assays were performed as follows. Eighty microlitres of test solution (calibrator) was loaded by micropipette into the sample loading port (42) of each device and allowed to incubate at room temperature for 20 minutes. A wash step was performed by applying 1.5 ml phosphate buffered saline pH7.4 containing 0.1% tween to the loading port and removing the same volume from the exit port by micropipette. A 100 ul aliquot of TMB was subsequently added to the loading port and an additional 100 ul fluid removed from the exit port. Assay signals were allowed to develop for 10 minutes, monitoring Transmission at 632 nm by means of the opto-electonic reader rig.

Transmission signals at 632 nm measured after 10 minutes development were normalised to the transmission signal during the PBS wash step and converted to Normalised Assay Signals as follows:

$$Normalised \% Transmission = \frac{Transmission at 632 nm after 10 minutes}{Transmission at 632 nm during PBS wash step} \times 100$$

$$Normalised \% Assay Signal = 100 - Normalised \% Transmission$$

Results are shown below.

| | | | | |
|---|---|---|---|---|
| Pi-GST concentration (ng/ml) | 0 | 2.5 | 10 | 40 |
| Normalised % Assay Signal | 7 | 12 | 15 | 39 |

### 5. Pi-GST Assays Using a Prototype Capillary Device (Absorbent Pad Method).

Pi-GST assays were performed in prototype devices using pi-GST ELISA kit reagents (Argutus Medical) in conjunction with a biotinylated capture antibody (Fleet Bioprocessing) as described below. The outlet of the capillary devices were modified mechanically to accommodate a multilayer absorbent pad.

Prototype devices were prepared for assay use as follows.

Fin components (FIG. 4) were prepared by applying a coating of streptavidin as described in example 4.

Modified capillary devices were prepared for assay use as described in example 4.

Devices were assembled by sealing the moulded capillary channels using self-adhesive tape and inserting a streptavidin-coated fin component into the central slot of the capillary device. Absorbent pads, consisting of a single layer of Ahlstrom 8964 Conjugate Pad and two layers of Ahlstrom 320 Absorbent Pad materials measuring approximately 5 mm diameter, 10 mm×20 mm and 10 mm×35 mm respectively, were cut to size and fitted into the machined recess overlying and adjoining the outlet of the capillary device.

Assembled devices were slotted into a purpose-built electronic spectrophotometric rig, containing an LED light source and photodiode detector, with PC-based user-interface and processing software. Transmission at 632 nm was monitored across the optical read/capture zone (SMZ) and the data recorded.

Test solutions were prepared by dilution of pi-GST kit calibrators in kit sample diluent to concentrations between 0 ng/ml and 40 ng/ml.

Assays were performed as follows. Forty-five microlitres of test solution (calibrator) was loaded by micropipette into the sample loading port (42) of each device and allowed to incubate at room temperature for 20 minutes. A wash step was performed by applying 1.5 ml phosphate buffered saline pH7.4 containing 0.1% tween to the loading port, followed by 100 ul TMB. Assay signals were allowed to develop for 10 minutes, monitoring Transmission at 632 nm by means of the opto-electonic reader rig.

Transmission signals at 632 nm were measured after 10 minutes development and converted to Normalised Assay Signals as described in example 4. Results are shown below.

| | | | | |
|---|---|---|---|---|
| Pi-GST concentration (ng/ml) | 0 | 2.5 | 10 | 40 |
| Normalised % Assay Signal | 12 | 19 | 25 | 45 |

### 6. Pi-GST Assays Using a Prototype Capillary Device.

Pi-GST assays were performed using prototype sample testing devices comprising 3 consecutive fluid application regions connected via a single capillary channel to a serpentine capture and signal measurement (optical) zone, followed by a moulded twin-spiral capillary passage acting as a fixed capacity fluid sump.

Assay reagents, based on those contained in a pi-GST ELISA kit (EKF Diagnostics) were prepared by freeze-drying a mixture of biotinylated ELISA “capture” antibody (66 ng per reaction), horseradish peroxidase conjugated ELISA “signal” antibody (24 ng per reaction) and selected cryoprotectants in individual moulded “reagent cups”.

Prototype devices were prepared for assay use by inserting a streptavidin-coated fin component (as described above) into the central slot of the device, and inserting a moulded reagent cup containing freeze-dried assay reagents into a reciprocally-shaped recess located above the first fluid application region.

Assembled devices were positioned in a purpose-built electronic spectrophotometric rig, containing an LED light source and photodiode detector, with PC-based user-interface and processing software. Transmission at 632 nm was monitored across the optical read/capture zone (SMZ) and the data recorded.

Test solutions were prepared by dilution of pi-GST kit calibrators in a 4:1 mixture of kit sample diluent and urine stabilising buffer (EKF Diagnostics) to concentrations between 0 ng/ml and 200 ng/ml.

Assays were performed as follows. Sixty-five microlitres (ul) of test solution (calibrator) was loaded into each device by micropipette via the reagent cup, which reconstituted the reagents and the mixture flowed into the capillary passage. After incubation at room temperature for 15 minutes, 500 ul phosphate buffered saline pH7.4 containing 0.1% tween was added to the second entry port. After the wash buffer had flowed into the device, a 300 ul aliquot of TMB (3,3,5,5-tetramethylbenzidine) was added to the third application region. No external propulsive force was applied to cause the fluids to flow into the test device. Assay signals were allowed to develop for 10 minutes after addition of TMB. Transmission across the capture/signal measurement zone was monitored by means of the opto-electronic reader rig, and when fluid flow in the capillary had ceased the rate of signal generation was automatically measured.

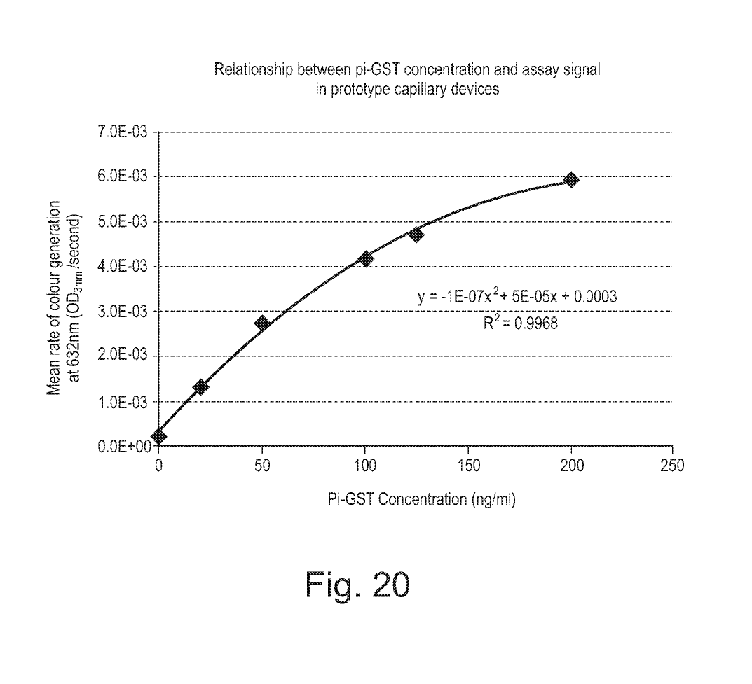

The following results show a clear dose-response relationship between pi-GST concentration and assay signal (rate of generation of blue colour at 632 nm) (FIG. 20).

| Pi-GST concentration | Mean rate of blue colour generation |
|---|---|
| (ng/ml) | (OD_{3 mm} per second) |
| 0 | 2.023 × 10⁻⁴ |
| 20 | 1.326 × 10⁻³ |
| 50 | 2.741 × 10⁻³ |
| 100 | 4.161 × 10⁻³ |
| 125 | 4.710 × 10⁻³ |
| 200 | 5.919 × 10⁻³ |

### Detailed Description of a Device According to the Invention

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A sample testing device for performing a heterogeneous assay, wherein the device comprises:
(i) a capillary passage having a lumen;
(ii) a combined capture and signal measurement zone fluidly connected to the a capillary passage; and
(iii) an optical pathway across the combined capture and signal measurement zone; wherein the combined capture and signal measurement zone includes a plurality of elongate fins projecting substantially perpendicularly from a base, where each elongate fin has a length that is substantially parallel to the base, the elongate fins being arranged so that:
the lengths of the plurality of elongate fins are substantially parallel to one another;
the plurality of elongate fins are aligned along a line that is substantially perpendicular to the lengths of the fins; and
the lengths of the plurality of elongate fins are substantially perpendicular to said optical pathway;
said plurality of elongate fins permitting optical transmission therethrough along said optical pathway and defining a plurality of fluidic channels therebetween along the base for receiving fluid from said capillary passage.

2-75. (canceled)
